(19) United States
(12) Patent Application Publication
(10) Pub. No.: US 2026/0250219 A1
(43) Pub. Date: Aug. 27, 2026

(54) Production of P-Xylene by Liquid-Phase Isomerization in the Presence of C9+ Aromatic Hydrocarbons and Separation Thereof

(71) Applicant: ExxonMobil Chemical Patents Inc., Baytown, TX (US)

(72) Inventors: Paul Podsiadlo, Spring, TX (US); Robert G. Tinger, Friendswood, TX (US); Xiaobo Zheng, Houston, TX (US)

(21) Appl. No.: 18/872,400

(22) PCT Filed: May 16, 2023
(86) PCT No.: PCT/US2023/022310

§ 371 Date: Dec. 6, 2024

Related U.S. Application Data
(60) Provisional application No. 63/351,905, filed on Jun. 14, 2022.

(51) Int. Cl. C07C 5/22; C07C 7/04

(52) U.S. Cl. CPC C07C 5/222; C07C 7/04

(57) ABSTRACT

A feed mixture comprising one or more xylene isomers and C9+ aromatic hydrocarbons may be separated in a first distillation column to obtain a first overhead stream comprising m-xylene and/or p-xylene and a first bottoms stream comprising o-xylene and C9+ aromatic hydrocarbons. The first bottoms stream may be optionally isomerized through liquid-phase isomerization and fed to a second distillation column to obtain a second overhead stream comprising at least o-xylene and a second bottoms stream comprising C9+ aromatic hydrocarbons. The second overhead stream may be optionally isomerized through liquid-phase isomerization. Liquid-phase isomerization may occur upon at least one of the first bottoms stream, the second overhead stream, an optional side stream obtained from the first or second distillation columns, or within the first or second distillation columns, such that the second overhead stream or an isomerized second overhead stream is rich in p-xylene.

### CROSS-REFERENCE TO RELATED APPLICATION

This application claims priority to and the benefit of U.S. Provisional Application No. 63/351,905 having a filing date of Jun. 14, 2022, the disclosure of which is incorporated herein by reference in its entirety.

### FIELD

The present disclosure relates to isomerization of C8 aromatic hydrocarbons and, more particularly, isomerization of C8 aromatic hydrocarbons in the presence of C9+ aromatic hydrocarbons under liquid-phase isomerization conditions and separation of p-xylene therefrom.

### BACKGROUND

Worldwide production capacity of p-xylene from various industrial sources is about 40 million tons per year. p-Xylene is a valuable chemical feedstock that may be obtained from C8+ aromatic hydrocarbon mixtures, primarily for conversion into 1,4-benzenedicarboxylic acid (terephthalic acid), which may be used in synthetic textiles, bottles, and plastic materials among other industrial applications. Other xylene isomers experience considerably lower, though still significant demand. m-Xylene, for instance, may be utilized as an aviation gas blending component.

C8+ aromatic hydrocarbon mixtures (e.g., o-, m-, and/or p-xylene isomers, as well as ethylbenzene and heavier aromatic hydrocarbons) may be produced through various processes, such as alkylation of lower aromatic hydrocarbons (e.g., benzene and/or toluene), transalkylation, toluene disproportionation, catalytic reforming, isomerization, cracking (e.g., steam or catalytic cracking), and the like. Alkylation of lower aromatic hydrocarbons with methanol and/or dimethyl ether under zeolite catalyst promotion may be a particularly effective and advantageous route for producing p-xylene at relatively high selectivity relative to o- and m-xylene, as described in, for example, U.S. Patent Application Publication 20200308085 and International Patent Application Publication WO/2020/197888, each of which is incorporated herein by reference.

After at least partially separating p-xylene from other C8+ aromatic hydrocarbons, a raffinate stream lean in p-xylene may be obtained. Such raffinate streams may be isomerized to form additional p-xylene and then undergo further separation to isolate the additional p-xylene that has been produced. Conventionally, such isomerization processes have been conducted using vapor-phase isomerization, which is a very energy-intensive process. There has been recent progress in conducting isomerization of xylene isomers by liquid-phase isomerization, which is typically much less energy-intensive. Although typically less energy-intensive, the presence of ethylbenzene and/or C9+ aromatic hydrocarbons during liquid-phase isomerization may generate unwanted byproducts and result in p-xylene loss or complicated separation thereof.

### SUMMARY

In some aspects, the present disclosure provides processes comprising: (I) introducing a feed mixture comprising one or more xylene isomers and C9+ aromatic hydrocarbons into a first distillation column at a feed location thereon, the first distillation column optionally containing a liquid-phase isomerization catalyst loaded therein; (II) optionally, contacting a portion of the feed mixture with the liquid-phase isomerization catalyst in the first distillation column under liquid-phase isomerization conditions; (III) obtaining from the first distillation column a first overhead stream comprising m-xylene, p-xylene, or any combination thereof, a first bottoms stream comprising o-xylene and C9+ aromatic hydrocarbons, and an optional first side stream; (IV) optionally, a) obtaining the first side stream from the first distillation column, b) conducting liquid-phase isomerization of the first side stream external to the first distillation column under liquid-phase isomerization conditions in the presence of a liquid-phase isomerization catalyst to produce an isomerized first side stream, and c) returning at least a portion of the isomerized first side stream to the first distillation column; (V) optionally, conducting liquid-phase isomerization upon at least a portion of the first bottoms stream external to the first distillation column under liquid-phase isomerization conditions in the presence of a liquid-phase isomerization catalyst to produce an isomerized first bottoms stream; (VI) feeding at least a portion of the first bottoms stream and/or at least a portion of the isomerized first bottoms stream to a second distillation column optionally containing a liquid-phase isomerization catalyst loaded therein; (VII) optionally, contacting a portion of the first bottoms stream with the liquid-phase isomerization catalyst in the second distillation column under liquid-phase isomerization conditions; (VIII) obtaining from the second distillation column a second overhead stream comprising at least o-xylene, a second bottoms stream comprising C9+ aromatic hydrocarbons, and an optional second side stream; (IX) optionally, a) obtaining the second side stream from the second distillation column, b) conducting liquid-phase isomerization of the second side stream external to the second distillation column under liquid-phase isomerization conditions in the presence of a liquid-phase isomerization catalyst to produce an isomerized second side stream, and c) returning at least a portion of the isomerized second side stream to the second distillation column; and (X) optionally, conducting liquid-phase isomerization upon at least a portion of the second overhead stream external to the second distillation column under liquid-phase isomerization conditions in the presence of a liquid-phase isomerization catalyst to produce an isomerized second overhead stream; wherein liquid-phase isomerization is carried out in at least one of (II), (IV), (V), (VII), (IX), or (X), and the second overhead stream and/or the isomerized second overhead stream is rich in p-xylene.

In some aspects, the present disclosure provides processes comprising: (A) providing a feed mixture comprising one or more xylene isomers and C9+ aromatic hydrocarbons, o-xylene comprising at least a majority of the one or more xylene isomers, and the feed mixture being lean in ethylbenzene; (B) optionally, conducting liquid-phase isomerization upon at least a portion of the feed mixture under liquid-phase isomerization conditions in the presence of a liquid-phase isomerization catalyst to produce an isomerized feed mixture; (C) introducing the feed mixture and/or at least a portion of the isomerized feed mixture into a distillation column at a feed location thereon, the distillation column optionally containing a liquid-phase isomerization catalyst loaded therein; (D) optionally, contacting a portion of the feed mixture with the liquid-phase isomerization catalyst in the distillation column under liquid-phase isomerization conditions; (E) obtaining from the distillation column an overhead stream comprising at least o-xylene, a bottoms stream comprising C9+ aromatic hydrocarbons, and an optional side stream; (F) optionally, a) obtaining the side stream from the distillation column, b) conducting liquid-phase isomerization of the side stream external to the distillation column under liquid-phase isomerization conditions in the presence of a liquid-phase isomerization catalyst to produce an isomerized side stream, and c) returning at least a portion of the isomerized side stream to the distillation column; (G) optionally, conducting liquid-phase isomerization upon at least a portion of the overhead stream external to the distillation column under liquid-phase isomerization conditions in the presence of a liquid-phase isomerization catalyst to produce an isomerized overhead stream; wherein liquid-phase isomerization is carried out in at least one of (B), (D), (F), or (G), and the overhead stream and/or the isomerized overhead stream is rich in p-xylene.

These and other features and attributes of the disclosed methods and compositions of the present disclosure and their advantageous applications and/or uses will be apparent from the detailed description which follows.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

To assist one of ordinary skill in the relevant art in making and using the subject matter hereof, reference is made to the appended drawings, wherein:

FIG. 1 is a block diagram of a system and process for xylene separation and liquid-phase isomerization according to the present disclosure, in which a feed mixture containing C9+ aromatic hydrocarbons and optionally ethylbenzene may be processed.

FIGS. 2-5 show block diagrams of system and process configurations similar to system and process 100 shown in FIG. 1, in which liquid-phase isomerization takes place at one location.

FIG. 6 is a block diagram of a system and process for xylene separation and liquid-phase isomerization according to the present disclosure, in which a feed mixture lean in ethylbenzene and containing C9+ aromatic hydrocarbons may be further processed.

FIGS. 7-9 show block diagrams of system and process configurations similar to system and process 600 shown in FIG. 6, in which liquid-phase isomerization takes place at one location.

### DETAILED DESCRIPTION

The present disclosure relates to isomerization of C8 aromatic hydrocarbons and, more particularly, isomerization of C8 aromatic hydrocarbons in the presence of C9+ aromatic hydrocarbons under liquid-phase isomerization conditions and separation of p-xylene therefrom.

### Definitions

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process may be described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described. However, various steps may occur non-sequentially and/or simultaneously rather than expressly in the order listed.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite articles “a” or “an” shall mean “at least one” unless specified to the contrary or the context clearly indicates otherwise. Thus, for example, embodiments using “a fractionation column” include embodiments where one, two or more fractionation columns are used, unless specified to the contrary or the context clearly indicates that only one fractionation column is used.

As used herein, the term “consisting essentially of” means a composition, feed, stream or effluent that includes a given component or group of components at a concentration of at least about 60 wt %, preferably at least about 70 wt %, more preferably at least about 80 wt %, more preferably at least about 90 wt %, or still more preferably at least about 95 wt %, based on the total weight of the composition, feed, stream or effluent.

The following abbreviations may be used herein for the sake of brevity: RT is room temperature (and is 23° C. unless otherwise indicated), kPag is kilopascal gauge, psig is pound-force per square inch gauge, psia is pounds-force per square inch absolute, and WHSV is weight hourly space velocity.

As used herein, “wt %” means percentage by weight, “vol %” means percentage by volume, “mol %” means percentage by mole, “ppm” means parts per million, and “ppm wt” and “wppm” are used interchangeably to mean parts per million on a weight basis. All concentrations herein are expressed on the basis of the total amount of the composition in question. All ranges expressed herein should include both end points as two specific embodiments unless specified or indicated to the contrary.

Nomenclature of elements and groups thereof used herein are pursuant to the Periodic Table used by the International Union of Pure and Applied Chemistry after 1988. An example of the Periodic Table is shown in the inner page of the front cover of Advanced Inorganic Chemistry, 6^{th} Edition, by F. Albert Cotton et al. (John Wiley & Sons, Inc., 1999).

As used herein, the term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of such at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn-hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

As used herein, an “aromatic hydrocarbon” is a hydrocarbon comprising an aromatic ring in the molecular structure thereof. An aromatic compound may have a cyclic cloud of pi electrons meeting the Hückel rule. A “non-aromatic hydrocarbon” means a hydrocarbon other than an aromatic hydrocarbon.

As used herein, the term “lower aromatic hydrocarbons” refers to benzene, toluene, or a mixture of benzene and toluene.

An “effluent” or a “feed” is sometimes also called a “stream” in this disclosure. Where two or more streams are shown to form a joint stream and then supplied into a vessel, it should be interpreted to include alternatives where the streams are supplied separately to the vessel where appropriate. Likewise, where two or more streams are supplied separately to a vessel, it should be interpreted to include alternatives where the streams are combined before entering into the vessel as joint stream(s) where appropriate. Furthermore, a single stream may be split into two or more separate streams and provided to different locations.

As used herein, the term “liquid-phase” means reaction conditions in which aromatic hydrocarbons present in a reactor are substantially in a liquid state. “Substantially in liquid-phase” means ≥about 90 wt %, preferably ≥about 95 wt %, preferably ≥about 99 wt %, and preferably the entirety of the aromatic hydrocarbons, is in a liquid-phase.

As used herein, the term “vapor-phase” means reaction conditions in which aromatic hydrocarbons present in a reactor are substantially in a vapor state. “Substantially in vapor-phase” means ≥about 90 wt %, preferably ≥about 95 wt %, preferably ≥about 99 wt %, and preferably the entirety of the aromatic hydrocarbons, is in a vapor-phase.

As used herein, the term “alkylation” means a chemical reaction in which an alkyl group is transferred to an aromatic ring as a substitute group thereon from an alkyl group source compound, such as an alkylating agent. “Methylation” means alkylation in which the transferred alkyl group is a methyl group. Thus, methylation of benzene can produce toluene, xylenes, trimethylbenzenes, and the like; and methylation of toluene can produce xylenes, trimethylbenzenes, and the like.

As used herein, the term “methylated aromatic hydrocarbon” means an aromatic hydrocarbon comprising at least one methyl group and only methyl group(s) attached to the aromatic ring(s) therein. Examples of methylated aromatic hydrocarbons include toluene, xylenes, trimethylbenzenes, tetramethylbenzenes, pentamethylbenzene, hexamethylbenzene, methylnaphthalenes, dimethylnaphthalenes, trimethylnaphthalenes, tetramethylnaphthalenes, and the like.

As used herein, the term “molecular sieve” means a crystalline or semi-crystalline substance, such as a zeolite, with pores of molecular dimensions that permit the passage of molecules below a certain threshold size.

“Crystallite” means a crystalline grain of a material. Crystallites with microscopic or nanoscopic size can be observed using microscopes such as transmission electron microscope (“TEM”), scanning electron microscope (“SEM”), reflection electron microscope (“REM”), scanning transmission electron microscope (“STEM”), and the like. Crystallites may aggregate to form a polycrystalline material. An agglomerate particle comprising multiple crystallites may be present in a material in some cases.

As used herein, the term “rich” or “enriched,” when describing a component in a stream or feed, means that the stream or feed comprises the component at a concentration higher than a source material from which the stream is derived. As used herein, the term “depleted” or “lean,” when describing a component in a stream or feed, means that the stream or feed comprises the component at a concentration lower than a source material from which the stream or feed is derived.

Unless otherwise specified herein, any stream or feed that is “rich” in a particular component may “consist of” or “consist essentially of” that component. A “rich” component of a feed or stream may comprise a majority component of the feed or stream in comparison to other components.

As used herein, the term “overhead stream” refers to a vapor stream that is removed from a top portion of a distillation column.

As used herein, the term “bottoms stream” refers to a liquid stream that is removed from a lower portion of a distillation column.

As used herein, the term “lower stream” refers to a vapor stream or a liquid stream that is not an overhead stream and is removed from a location other than a top portion of a distillation column. A “lower stream” may be a side stream or a bottoms stream. Depending on the vertical location from which it is removed, a side stream may be a vapor stream, a liquid stream, or a combination thereof.

Unless otherwise specified herein, any stream or feed that is “lean” in a particular component may be “free of” or “substantially free of” that component. “Essentially free of” and “substantially free of,” as interchangeably used herein, mean that a composition, feed, stream or effluent comprises a given component at a concentration of at most about 10 wt %, preferably at most about 8 wt %, more preferably at most about 5 wt %, more preferably at most about 3 wt %, and still more preferably at most about 1 wt %, based on the total mass of the composition, feed, stream or effluent in question.

In this disclosure, o-xylene means 1,2-dimethylbenzene, m-xylene means 1,3-dimethylbenzene, and p-xylene means 1,4-dimethylbenzene. Herein, the generic term “xylene(s) or xylene isomer(s),” either in singular or plural form, collectively means one of or any mixture of two through four of p-xylene, m-xylene, and o-xylene at any proportion thereof, and/or ethylbenzene. In the disclosure herein, ethylbenzene is to be considered a xylene isomer. Thus, a mixture of xylene isomers may comprise or consist essentially of one or more of o-xylene, m-xylene, p-xylene, and ethylbenzene. A stream containing xylene isomers may be lean in p-xylene or rich in p-xylene, depending on the location and processing conditions from which the stream is drawn, as explained further herein.

A stream or feed that is lean in one component may be rich in another component. For example, a stream lean in p-xylene may be rich in o-xylene and/or m-xylene and/or C9+ aromatic hydrocarbons.

### Liquid-Phase Isomerization of a Feed Mixture Containing C9+ Aromatic Hydrocarbons

As discussed above, it may be desirable to conduct isomerization of a raffinate stream obtained following separation of p-xylene from a feed mixture containing C8+ aromatic hydrocarbons. The isomerization may form additional p-xylene from other C8 aromatic hydrocarbons and promote more effective utilization of the feed mixture. Isomerization of this type has typically been conducted using vapor-phase isomerization, which is a very energy-intensive process. Advancements in liquid-phase isomerization of C8 aromatic hydrocarbons may offer benefits over vapor-phase isomerization, such as decreasing energy input requirements and minimal formation of byproducts. However, the presence of C9+ aromatic hydrocarbons in a mixed aromatic hydrocarbon feed mixture may generate unwanted byproducts and result in p-xylene loss and/or complicated separation thereof. Description of exemplary liquid-phase isomerization processes, conditions, and catalysts may be found in, for example, U.S. Patent Application Publications 2011/0319688; 2012/0108867; 2013/0274532; 2014/0023563; and 2015/0051430, the relevant contents of which are incorporated herein by reference. Additional details concerning liquid-phase isomerization processes, conditions, and catalysts are provided herein.

Catalysts effective for isomerizing xylene isomers under liquid-phase conditions to produce additional p-xylene may frequently act upon C9+ aromatic hydrocarbons as well and result in byproduct formation. The present disclosure provides advantaged processes for producing and separating p-xylene from feed mixtures also containing C9+ aromatic hydrocarbons, in which liquid-phase isomerization may be conducted effectively in the presence of the C9+ aromatic hydrocarbons using a suitable liquid-phase isomerization catalyst. Advantageously, liquid-phase isomerization catalysts comprising a zeolite having a MEL framework (e.g., ZSM-11) may readily promote isomerization of xylene isomers under liquid-phase isomerization conditions to produce an equilibrium mixture of xylenes from a stream lean in p-xylene. Surprisingly, zeolite catalysts having a MEL framework exhibit relatively low reactivity toward C9+ aromatic hydrocarbons and lead to minimal byproduct formation when contacted therewith. This benefit addresses a significant difficulty otherwise associated with conducting liquid-phase isomerization of feed mixtures containing significant amounts of C9+ aromatic hydrocarbons. In addition, the various advantaged process configurations disclosed herein allow considerable flexibility to be realized in the locations where liquid-phase isomerization takes place. That being the case, the advantaged process configurations disclosed herein may even facilitate use of zeolite catalysts that are less tolerant to the presence of C9+ aromatic hydrocarbons, provided that liquid-phase isomerization is conducted at a suitable process location. For example, zeolite catalysts having a MFI framework may also be used during liquid-phase isomerization in the processes disclosed herein in some instances.

Further advantages of the present disclosure may include lowering the separation burden (column specification) for separating p-xylene from C9+ aromatic hydrocarbons and other xylene isomers in a distillation column. By lowering the separation burden, further energy efficiencies may be realized in addition to those resulting from conducting liquid-phase isomerization (as opposed to vapor-phase isomerization) upon the resulting raffinate stream lean in p-xylene. More specifically, since liquid-phase isomerization may be conducted effectively in the presence of C9+ aromatic hydrocarbons in the present disclosure, complete removal of xylene isomers from the raffinate stream is not required, as the xylene isomers may be subsequently isomerized to produce additional p-xylene. In addition, a decreased separation burden may potentially lower capital equipment costs by facilitating use of smaller distillation columns. C9+ aromatic hydrocarbons may be separated from the process stream in the present disclosure and undergo further conversion into one or more additional value products, if desired.

The advantaged separation processes of the present disclosure may further tolerate feed mixtures containing significant quantities of ethylbenzene by conducting early-stage separation of the ethylbenzene from the raffinate stream. Moreover, the liquid-phase isomerization catalysts and liquid-phase isomerization conditions of the present disclosure also do not lead to significant generation of ethylbenzene and other byproducts when further processing the raffinate stream to generate additional p-xylene, which might otherwise complicate further separation of p-xylene in a p-xylene recovery unit. To address ethylbenzene and other byproducts that may build in concentration during operation, the processes disclosed herein may further incorporate vapor-phase isomerization to mitigate components that may not be effectively converted using liquid-phase isomerization, which may afford further quantities of p-xylene. By coupling vapor-phase isomerization to a liquid-phase isomerization process according to the disclosure herein, the overall energy input requirements may be decreased in comparison to that of processing by the raffinate stream by vapor-phase isomerization alone. Additional details and further process advantages are discussed in the description that follows.

Before discussing more particular aspects and advantages of the present disclosure in further detail, the processes of the present disclosure will be described with reference to the drawings. In the interest of brevity, common reference characters are used in the drawings to describe elements having similar structure and function in various system and process configurations.

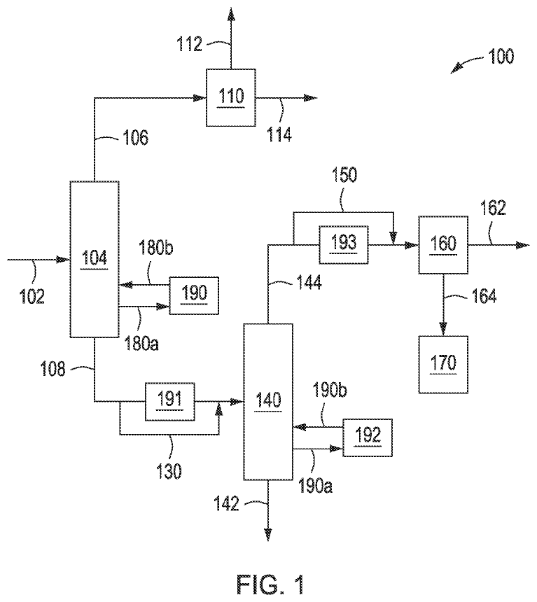

FIG. 1 is a block diagram of a system and process for xylene separation and liquid-phase isomerization according to the present disclosure, in which a feed mixture containing C9+ aromatic hydrocarbons and optionally ethylbenzene may be processed. In system and process 100, feed mixture 102, which comprises one or more xylene isomers, C9+ aromatic hydrocarbons, and optionally ethylbenzene, is introduced to distillation column 104. Feed mixture 102 may be rich in p-xylene or lean in p-xylene, depending on the source from which feed mixture 102 is received and how it has been previously processed. Upon separating feed mixture 102 in distillation column 104, overhead stream 106 and bottoms stream 108 may be produced. The separation to afford overhead stream 106 and bottoms stream 108 may be facilitated by virtue of the differing boiling points of the xylene isomers (o-xylene, m-xylene, and p-xylene have normal boiling points of 144° C., 139° C., and 138° C., respectively; and ethylbenzene has a normal boiling point of 136° C.). Facilitated by the differing boiling points, overhead stream 104 may comprise or consist essentially of lower-boiling components in feed mixture 102 (m-xylene, p-xylene, and ethylbenzene, if present), and at least a majority of higher-boiling components in feed mixture 102 (o-xylene and C9+ aromatic hydrocarbons) may localize within bottoms stream 108. As described further below, these components may also form within or adjacent to distillation column 104. Although bottoms stream 108 may comprise primarily o-xylene and C9+ aromatic hydrocarbons, it is not a requirement that complete resolution (separation) of m-xylene and/or p-xylene from o-xylene and C9+ aromatic hydrocarbons take place, since subsequent separation of p-xylene may take place after subsequent processing of bottoms stream 108 by liquid-phase isomerization according to the disclosure herein. Preferably, bottoms stream 108 may be substantially free of ethylbenzene to facilitate the liquid-phase isomerization and subsequent separation of p-xylene. Additional details regarding suitable locations at which the liquid-phase isomerization may be conducted and the composition of various streams obtained following liquid-phase isomerization are provided hereinafter.

Liquid-phase isomerization in system and process 100 may take place in one or more locations under liquid-phase isomerization conditions in the presence of a liquid-phase isomerization catalyst. In one example, distillation column 104 may contain a liquid-phase isomerization catalyst loaded therein, and liquid-phase isomerization may take place upon contacting at least a portion of feed mixture 104 with the liquid-phase isomerization catalyst within distillation column 104. In another alternative, side stream 180a may be removed from distillation column 102, and liquid-phase isomerization may take place external to distillation column 104 in liquid-phase isomerization unit 190. Isomerized side stream 180b may then be returned to distillation column 104, wherein isomerized side stream 180b may be returned to the same location from which side stream 180a was drawn or to a different location. When returned to a different location, isomerized side stream 180b may be returned to a location above or below a vertical position from which side stream 180a was drawn. Likewise, isomerized side stream 180b may be returned to a location above or below a feed location where feed mixture 102 is introduced to distillation column 104. Optionally, liquid-phase isomerization does not occur in distillation column 104 and side stream 180a is not withdrawn and isomerized, in which case liquid-phase isomerization takes place further downstream, as discussed further hereinbelow. Thus, depending on various process considerations, a liquid-phase isomerization catalyst may not be present in distillation column 104 and/or liquid-phase isomerization unit 190 may not be fluidly connected to distillation column 104.

Depending on the content of feed mixture 102 and/or whether liquid-phase isomerization is conducted in distillation column 104 or external to distillation column 104, the composition of overhead stream 106 may vary considerably. If feed mixture 102 is rich in p-xylene, or if liquid-phase isomerization is conducted in distillation column 104 or external to distillation column 104, overhead stream 106 may be correspondingly enriched in p-xylene. However, if feed mixture 102 is lean in p-xylene or if liquid-phase isomerization is not conducted in distillation column 104 or external to distillation column 104, overhead stream 106 may be lean in p-xylene, or p-xylene may even be substantially absent from overhead stream 106. If liquid-phase isomerization is conducted in distillation column 104 or external to distillation column 104, the liquid-phase isomerization may be conducted in a manner such that bottoms stream 108 contains an equilibrium or non-equilibrium distribution of xylene isomers. Preferably, the liquid-phase isomerization, if conducted in distillation column 104 or external to distillation column 104, may produce an equilibrium distribution of xylene isomers in bottoms stream 108, thereby obviating the need to conduct further liquid-phase isomerization further downstream, as discussed hereinafter. In addition to m-xylene and/or p-xylene, overhead stream 106 may further comprise ethylbenzene, if present in feed mixture 102.

Overhead stream 106 in FIG. 1 may be provided to p-xylene recovery unit 110, which may utilize adsorption chromatography, crystallization, or any combination thereof to afford first stream 112 rich in p-xylene and second stream 114 lean in p-xylene. Second stream 114 may comprise or consist essentially of m-xylene and ethylbenzene. If desired, second stream 114 may be further processed by vapor-phase isomerization under vapor-phase isomerization conditions in the presence of a vapor-phase isomerization catalyst to form additional p-xylene within an equilibrium mixture of xylene isomers, as described further below.

If bottoms stream 108 does not contain an equilibrium mixture of xylene isomers, liquid-phase isomerization may be conducted downstream from distillation column 104. Thus, in another example showing where liquid-phase isomerization may be conducted in system and process 100, bottoms stream 108 may be fed to liquid-phase isomerization unit 191 to conduct liquid-phase isomerization therein in the presence of a suitable liquid-phase isomerization catalyst under liquid-phase isomerization conditions, thereby forming an isomerized bottoms stream that is fed to distillation column 140. The liquid-phase isomerization in liquid-phase isomerization unit 191 may take place in the presence of the C9+ aromatic hydrocarbons. If liquid-phase isomerization of bottoms stream 108 is not desired or deemed necessary, liquid-phase isomerization unit 191 may be absent, or alternately, at least a portion of bottoms stream 108 may be diverted as bypass stream 130 before being provided to distillation column 140.

Bottoms stream 108 or an isomerized bottoms stream produced therefrom is fed to distillation column 140 and is separated into overhead stream 144 and bottoms stream 142. Bottoms stream 142 comprises or consists essentially of C9+ aromatic hydrocarbons, which are removed from the process stream and optionally further processed (not shown), such as through transalkylation or disproportionation to produce additional p-xylene.

Overhead stream 144 obtained from distillation column 140 may comprise at least o-xylene, which may originate from bottoms stream 108 or an isomerized bottoms stream produced therefrom. Other xylene isomers (e.g., m-xylene and/or p-xylene) originating from bottoms stream 108 may be present in overhead stream 144 if liquid-phase isomerization has been conducted upstream from distillation column 140, and/or if distillation column 104 did not completely separate m-xylene and/or p-xylene from feed mixture 102 into overhead stream 106.

In still another example, distillation column 140 may contain a liquid-phase isomerization catalyst loaded therein, and liquid-phase isomerization may take place upon contacting at least a portion of bottoms stream 108 with the liquid-phase isomerization catalyst within distillation column 140. In another alternative, side stream 190a may be removed from distillation column 140, and liquid-phase isomerization may take place external to distillation column 140 in liquid-phase isomerization unit 192. Isomerized side stream 190b may then be returned to distillation column 140, wherein isomerized side stream 190b may be returned to the same location from which side stream 190a was drawn or to a different location. When returned to a different location, isomerized side stream 190b may be returned to a location above or below a vertical position from which side stream 190a was drawn. Likewise, isomerized side stream 190b may be returned to a location above or below a feed location where bottoms stream 108 is introduced to distillation column 140. Optionally, liquid-phase isomerization does not occur in distillation column 140 and side stream 190a is not withdrawn and isomerized. If liquid-phase isomerization has taken place upstream from distillation column 140, as described above, liquid-phase isomerization within distillation column 140 and/or withdrawal and liquid-phase isomerization of side stream 190a may be omitted. Alternately, if liquid-phase isomerization in distillation column 140 and/or withdrawal and liquid-phase isomerization of side stream 190a do not take place, liquid-phase isomerization may take place further downstream, as discussed further below. Thus, depending on various process considerations, a liquid-phase isomerization catalyst may not be present in distillation column 140 and/or liquid-phase isomerization unit 192 may not be fluidly connected to distillation column 140.

Accordingly, similar to the discussion above, overhead stream 144 may be lean in p-xylene if liquid-phase isomerization has not been conducted upstream from distillation column 140, within distillation column 140, or within liquid-phase isomerization unit 192. In such cases, overhead stream 144 may comprise predominantly or consist essentially of o-xylene. If, however, liquid-phase isomerization has been conducted in any of these locations, overhead stream 144 may be rich in p-xylene and preferably comprise a mixture of xylene isomers. Preferably, overhead stream 144 may remain lean in ethylbenzene, since production rates for ethylbenzene may remain low under liquid-phase isomerization conditions.

If overhead stream 144 does not contain an equilibrium mixture of xylene isomers or if liquid-phase isomerization has not been conducted in an upstream location, liquid-phase isomerization may be conducted downstream from distillation column 140. Thus, in another example showing where liquid-phase isomerization may be conducted in system and process 100, overhead stream 144 may be fed to liquid-phase isomerization unit 193 to conduct liquid-phase isomerization therein in the presence of a suitable liquid-phase isomerization catalyst, thereby forming an isomerized overhead stream that may be subsequently processed. Optionally, if liquid-phase isomerization of overhead stream 144 is not desired or deemed necessary, liquid-phase isomerization unit 193 may be absent, or alternately, at least a portion of overhead stream 144 may be diverted as bypass stream 150.

Overhead stream 144 or an isomerized overhead stream produced therefrom may be fed to p-xylene recovery unit 160. p-Xylene recovery unit 160 may utilize a separation technique such as adsorption chromatography, crystallization, or any combination thereof to facilitate separation of overhead stream 144 or an isomerized overhead stream produced therefrom into first stream 162 that is rich in p-xylene and may consist essentially of p-xylene, and second stream 164, which may be lean in p-xylene or comprise p-xylene in combination with other xylene isomers. For example, second stream 164 may comprise predominantly other xylene isomers if separation in p-xylene recovery unit 160 takes place by adsorption chromatography, such as simulated moving bed chromatography. Conversely, second stream 164 may comprise p-xylene in combination with one or more other xylene isomers (e.g., a mixture of p-xylene with o-xylene and m-xylene) if separation takes place by crystallization in p-xylene recovery unit 160. Commercially available simulated moving bed chromatography processes suitable for use in p-xylene recovery unit 160 are available from Axens, a French corporation, as ELUXYL® technology, although any other simulated moving bed process may also be effectively utilized. Second stream 164 may undergo subsequent vapor-phase isomerization in vapor-phase isomerization unit 170 under vapor-phase isomerization conditions in the presence of a vapor-phase isomerization catalyst to promote additional formation of xylene therefrom. Optionally, second stream 114 may also be introduced to vapor-phase isomerization unit. Vapor-phase isomerization unit 170 may comprise a portion of a xylenes isomerization loop (not shown), which may further include a distillation column for separating xylene isomers from other aromatic hydrocarbons and a p-xylene recovery unit, which may utilize simulated moving bed chromatography or crystallization recovery technologies. Additional details regarding suitable vapor-phase isomerization conditions and vapor-phase isomerization catalysts are provided hereinbelow.

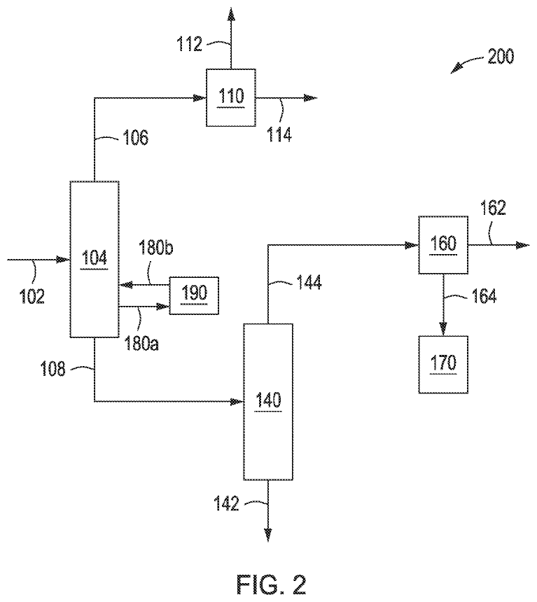

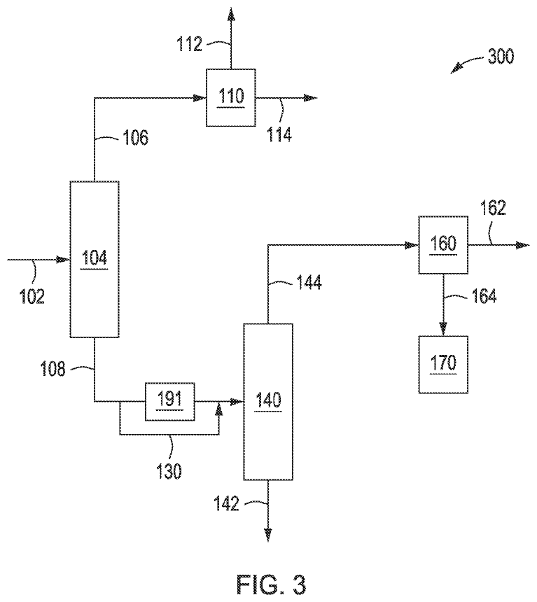

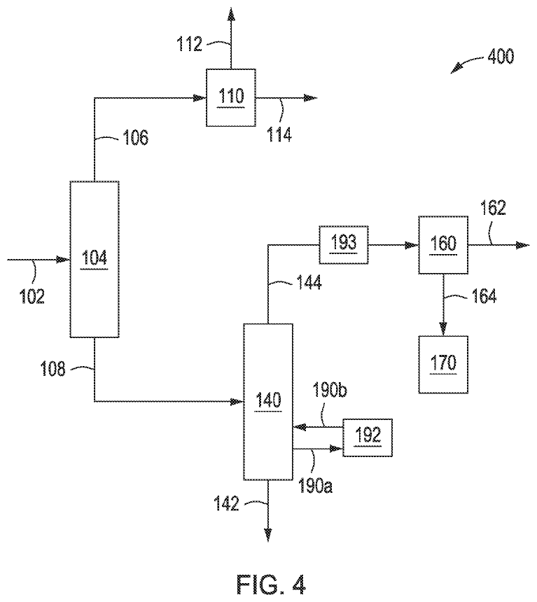

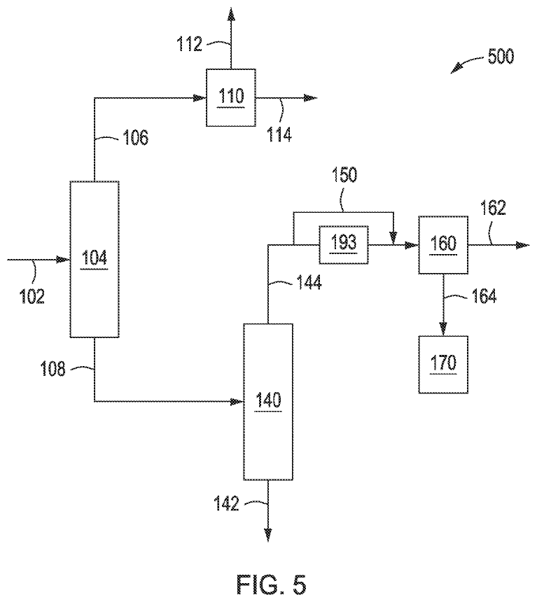

Preferably, the liquid-phase isomerization conducted at any of the various locations within system and process 100 may afford an equilibrium mixture of xylene isomers. Once an equilibrium mixture of xylene isomers has been formed, the concentration of p-xylene may not be increased through further isomerization until the equilibrium position has been altered, such as through withdrawing p-xylene from the process stream. Accordingly, more specific process configurations may feature liquid-phase isomerization conducted in one of the foregoing locations, such as within distillation column 100, external to distillation column 104 in liquid-phase isomerization unit 190, upon bottoms stream 108 within liquid-phase isomerization unit 191, within distillation column 140, external to distillation column 140 within liquid-phase isomerization unit 192, or upon overhead stream 144 within liquid-phase isomerization unit 193. FIGS. 2-5 show block diagrams of system and process configurations similar to system and process 100 shown in FIG. 1, in which liquid-phase isomerization takes place at one location, all but the latter of which may conduct the liquid-phase isomerization in the presence of C9+ aromatic hydrocarbons. FIG. 2 shows a block diagram of system and process configuration 200, in which liquid-phase isomerization takes place upon side stream 180a within liquid-phase isomerization unit 190. FIG. 3 shows a block diagram of system and process configuration 300, in which liquid-phase isomerization takes place upon bottoms stream 108 within liquid-phase isomerization unit 191. FIG. 4 shows a block diagram of system and process configuration 400, in which liquid-phase isomerization takes place upon side stream 190a within liquid-phase isomerization unit 192. FIG. 5 shows a block diagram of system and process configuration 500, in which liquid-phase isomerization takes place upon overhead stream 144 within liquid-phase isomerization unit 193.

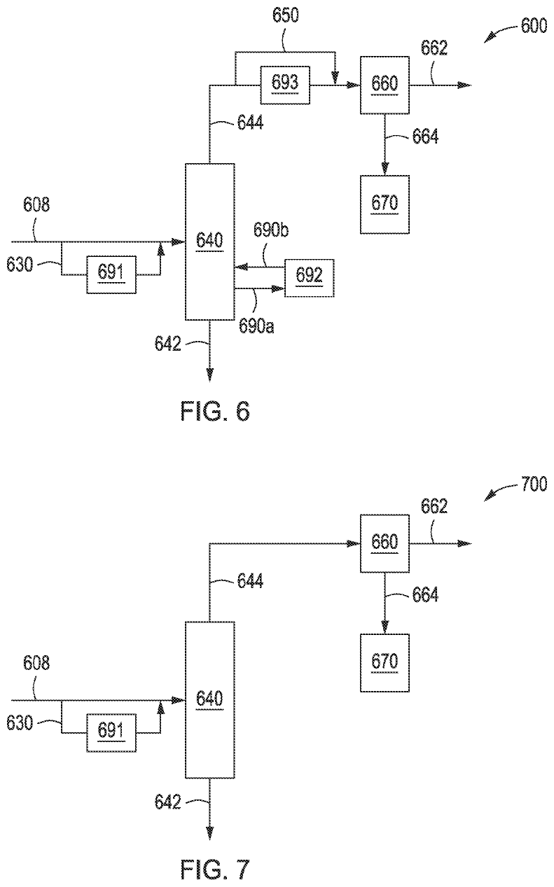

System and process configurations similar to those depicted in FIGS. 1-5 but omitting distillation column 104 are also feasible, provided that a feed mixture containing ethylbenzene below a threshold concentration that does not interfere with downstream separation is used. FIG. 6 is a block diagram of a system and process for xylene separation and liquid-phase isomerization according to the present disclosure, in which a feed mixture lean in ethylbenzene and containing C9+ aromatic hydrocarbons may be further processed. System and process 600 shown in FIG. 6 bears similarity to the portions of system and process 100 in FIG. 1 that are downstream from distillation column 104 and therefore may be better understood by reference thereto. Accordingly, the features of system and process 600 are only discussed in brief below.

Referring to FIG. 6, system and process 600 contains distillation column 640 that receives feed mixture 608 and promotes separation thereof into bottoms stream 642 and overhead stream 644. Feed mixture 608 may be lean in ethylbenzene (e.g., contain ethylbenzene below a specified amount that may enable further separations to be effectively conducted) and contain one or more xylene isomers and C9+ aromatic hydrocarbons. Feed mixture 608 may be rich in p-xylene and also contain at least one of m-xylene and p-xylene, or feed mixture 608 may be lean in p-xylene and contain at least one of m-xylene and o-xylene. For example, feed mixture 608 may be lean in ethylbenzene and comprise predominantly o-xylene and C9+ aromatic hydrocarbons in some instances.

Optionally, at least a portion of feed mixture 608 may undergo liquid-phase isomerization prior to being introduced to distillation column 640. As shown in FIG. 6, at least a portion of feed mixture 608 may be diverted to bypass stream 630, which provides the portion of feed mixture 608 to liquid-phase isomerization unit 691. An isomerized feed stream may be produced in liquid-phase isomerization unit 691 under liquid-phase isomerization conditions in the presence of a liquid-phase isomerization catalyst and subsequently fed to distillation column 640.

As referenced above, bottoms stream 642 and overhead stream 644 are obtained from distillation column 640. Bottoms stream 642 is rich in C9+ aromatic hydrocarbons and may consist essentially of C9+ aromatic hydrocarbons. The C9+ aromatic hydrocarbons within bottoms stream 642 may be removed from the process stream and further manipulated, if desired.

Optionally, distillation column 640 may contain a liquid-phase isomerization catalyst loaded therein, and liquid-phase isomerization may take place upon contacting at least a portion of feed mixture 608 with the liquid-phase isomerization catalyst within distillation column 640. In another alternative, side stream 690a may be removed from distillation column 640, and liquid-phase isomerization may take place external to distillation column 640 in liquid-phase isomerization unit 692 under liquid-phase isomerization conditions in the presence of a liquid-phase isomerization catalyst. Isomerized side stream 690b may then be returned to distillation column 640, wherein isomerized side stream 690b may be returned to the same location from which side stream 690a was drawn or to a different location. When returned to a different location, isomerized side stream 690b may be returned to a location above or below a vertical position from which side stream 690a was drawn. Optionally, liquid-phase isomerization does not occur in distillation column 640 and side stream 690a is not withdrawn and isomerized. If liquid-phase isomerization has taken place upstream from distillation column 640, (e.g., within liquid-phase isomerization unit 691, as described above), liquid-phase isomerization within distillation column 640 and/or withdrawal and liquid-phase isomerization of side stream 690a may be omitted. Alternately, if liquid-phase isomerization in distillation column 640 and/or withdrawal and liquid-phase isomerization of side stream 690a do not take place, liquid-phase isomerization may take place further downstream (e.g., upon overhead stream 644), as discussed further below. Thus, depending on various process considerations, a liquid-phase isomerization catalyst may not be present in distillation column 640 and/or liquid-phase isomerization unit 692 may not be fluidly connected to distillation column 640.

If overhead stream 644 does not contain an equilibrium mixture of xylene isomers or if liquid-phase isomerization has not been conducted in an upstream location, liquid-phase isomerization may be conducted downstream from distillation column 640. Thus, in another example showing where liquid-phase isomerization may be conducted in system and process 600, overhead stream 644 may be fed to liquid-phase isomerization unit 693 to conduct liquid-phase isomerization therein in the presence of a suitable liquid-phase isomerization catalyst under liquid-phase isomerization conditions, thereby forming an isomerized overhead stream that may be subsequently processed. Optionally, if liquid-phase isomerization of overhead stream 644 is not desired or deemed necessary, liquid-phase isomerization unit 693 may be absent, or alternately, at least a portion of overhead stream 644 may be diverted as bypass stream 650 without undergoing isomerization in liquid-phase isomerization unit 693.

Accordingly, overhead stream 644 may be rich or lean in p-xylene depending on whether feed mixture 608 is rich or lean in p-xylene and/or whether liquid-phase isomerization has been conducted prior to separating overhead stream 644. For example, overhead stream 644 may comprise predominantly or consist essentially of o-xylene in some examples.

Overhead stream 644 or an isomerized overhead stream produced therefrom is rich in p-xylene may be fed to p-xylene recovery unit 660. p-Xylene recovery unit 660 may utilize a separation technique such as adsorption chromatography, crystallization, or any combination thereof to facilitate separation of overhead stream 644 or an isomerized overhead stream produced therefrom into first stream 662 that is rich in p-xylene and may consist essentially of p-xylene, and second stream 664, which may be lean in p-xylene or comprise p-xylene in combination with other xylene isomers. For example, second stream 664 may comprise predominantly other xylene isomers if separation in p-xylene recovery unit 660 takes place by adsorption chromatography, such as simulated moving bed chromatography. Conversely, second stream 664 may comprise p-xylene in combination with one or more other xylene isomers (e.g., a mixture of p-xylene with o-xylene and m-xylene) if separation takes place by crystallization in p-xylene recovery unit 660. Second stream 664 may undergo subsequent vapor-phase isomerization in vapor-phase isomerization unit 670 under vapor-phase isomerization conditions in the presence of a vapor-phase isomerization catalyst to promote additional formation of p-xylene therefrom. Vapor-phase isomerization unit may comprise a portion of a xylenes loop (not shown), as discussed further above in reference to FIG. 1.

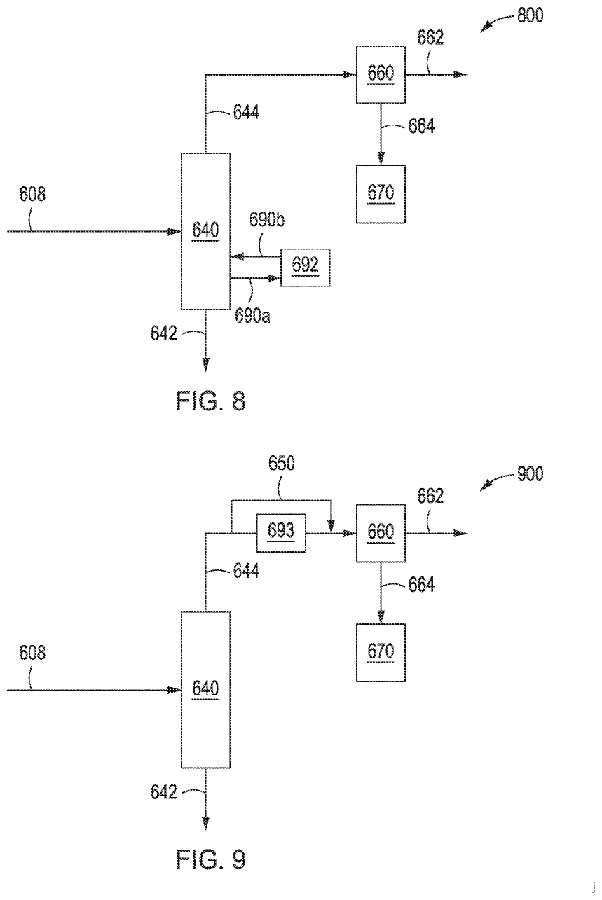

Preferably, the liquid-phase isomerization conducted at any of the various locations within system and process 600 may afford an equilibrium mixture of xylene isomers, and various configurations of system and process 600 may feature liquid-phase isomerization occurring in one location, such as upon feed mixture 608 within liquid-phase isomerization unit 691, within distillation column 640, external to distillation column 640 within liquid-phase isomerization unit 692, or upon overhead stream 644 within liquid-phase isomerization unit 693. FIGS. 7-9 show block diagrams of system and process configurations similar to system and process 600 shown in FIG. 6, in which liquid-phase isomerization takes place at one location. FIG. 7 shows a block diagram of system and process configuration 700, in which liquid-phase isomerization takes place upon feed mixture 608 within liquid-phase isomerization unit 691. FIG. 8 shows a block diagram of system and process configuration 800, in which liquid-phase isomerization takes place upon side stream 690a within liquid-phase isomerization unit 692. FIG. 9 shows a block diagram of system and process configuration 900, in which liquid-phase isomerization takes place upon overhead stream 644 within liquid-phase isomerization unit 693. In all but system and process 900, liquid-phase isomerization takes place in the presence of C9+ aromatic hydrocarbons.

Liquid-phase isomerization may be desirable for producing p-xylene from various feed mixtures that are lean in p-xylene. The system and process configurations disclosed herein advantageously may accommodate feed mixtures either rich or lean in p-xylene and optionally comprising varying amounts of ethylbenzene, the presence of which may complicate downstream separation of p-xylene if present in excess amounts. For example, in systems and processes similar to system and process 100, feed mixtures containing a high level of ethylbenzene may be effectively utilized, since the ethylbenzene may be drawn off into overhead stream 106 before conducting liquid-phase isomerization and subsequent separation of p-xylene. In systems and processes similar to system and process 600, feed mixtures having low levels of ethylbenzene or feed mixtures that may be refined to afford levels of ethylbenzene below a specified threshold may be effectively utilized without a separate distillation column to promote separation of ethylbenzene into an overhead stream. In either case, suitable feed mixtures may contain less than an equilibrium distribution of p-xylene with respect to other xylene isomers, such that production of p-xylene may be increased through conducting liquid-phase isomerization according to the disclosure herein. Any ethylbenzene that accumulates or is collected in the systems and processes disclosed herein may be subjected to subsequent vapor-phase isomerization to convert the ethylbenzene into other xylene isomers, since ethylbenzene undergoes relatively slow isomerization under liquid-phase isomerization conditions but may be readily isomerized under vapor-phase isomerization conditions. Conversely, the liquid-phase isomerization catalysts and liquid-phase isomerization conditions described herein further do not tend to produce significant quantities of ethylbenzene, thereby facilitating use thereof in the processes disclosed herein. Through utilization of the liquid-phase isomerization and separation processes disclosed herein, less energetic separation of p-xylene from a feed mixture may be realized. More effective utilization of the feed mixture may also be realized.

Advantageously and surprisingly, isomerization of xylene isomers under liquid-phase isomerization conditions may take place effectively in the presence of C9+ aromatic hydrocarbons, typically after separating ethylbenzene from a feed mixture. Surprisingly, liquid-phase isomerization catalysts specified herein may exhibit selectivity toward isomerizing C8 aromatic hydrocarbons in preference to C9+ aromatic hydrocarbons, which may limit byproduct formation resulting from exposure of the C9+ aromatic hydrocarbons to the liquid-phase isomerization conditions. Moreover, since liquid-phase isomerization of C8 aromatic hydrocarbons may take place effectively in the presence of C9+ aromatic hydrocarbons, it is not necessary to achieve complete separation of C8 aromatic hydrocarbons from C9+ aromatic hydrocarbons when separating ethylbenzene from a feed mixture. This reduced separation burden may lessen energy input requirements needed to separate and isomerize xylene isomers to form p-xylene according to the disclosure herein, as well as potentially lower capital equipment expenses by decreasing the size of distillation columns that are used during processing. A variety of system and process configurations suitable for conducting the liquid-phase isomerization in accordance with the foregoing are possible, as discussed in more detail above in reference to FIGS. 1-9. Moreover, although a liquid-phase isomerization catalyst having high selectivity toward isomerization of C8 aromatic hydrocarbons in preference of C9+ aromatic hydrocarbons may be desirable, the system and process configurations disclosed herein are also sufficiently flexible to accommodate liquid-phase isomerization catalysts that are less tolerant toward byproduct formation when contacting C9+ aromatic compounds. As such, a range of suitable liquid-phase isomerization catalysts may also be accommodated in the processes disclosed herein. Additional details regarding suitable liquid-phase isomerization catalysts and liquid-phase isomerization conditions are provided below.

Accordingly, the present disclosure provides isomerization and separation processes comprising: (I) introducing a feed mixture comprising one or more xylene isomers and C9+ aromatic hydrocarbons into a first distillation column at a feed location thereon, the first distillation column optionally containing a liquid-phase isomerization catalyst loaded therein; (II) optionally, contacting a portion of the feed mixture with the liquid-phase isomerization catalyst in the first distillation column under liquid-phase isomerization conditions; (III) obtaining from the first distillation column a first overhead stream comprising m-xylene, p-xylene, or any combination thereof, a first bottoms stream comprising o-xylene and C9+ aromatic hydrocarbons, and an optional first side stream; (IV) optionally, a) obtaining the first side stream from the first distillation column, b) conducting liquid-phase isomerization of the first side stream external to the first distillation column under liquid-phase isomerization conditions in the presence of a liquid-phase isomerization catalyst to produce an isomerized first side stream, and c) returning at least a portion of the isomerized first side stream to the first distillation column; (V) optionally, conducting liquid-phase isomerization upon at least a portion of the first bottoms stream external to the first distillation column under liquid-phase isomerization conditions in the presence of a liquid-phase isomerization catalyst to produce an isomerized first bottoms stream; (VI) feeding at least a portion of the first bottoms stream and/or at least a portion of the isomerized first bottoms stream to a second distillation column optionally containing a liquid-phase isomerization catalyst loaded therein; (VII) optionally, contacting a portion of the first bottoms stream with the liquid-phase isomerization catalyst in the second distillation column under liquid-phase isomerization conditions; (VIII) obtaining from the second distillation column a second overhead stream comprising at least o-xylene, a second bottoms stream comprising C9+ aromatic hydrocarbons, and an optional second side stream; and (IX) optionally, a) obtaining the second side stream from the second distillation column, b) conducting liquid-phase isomerization of the second side stream external to the second distillation column under liquid-phase isomerization conditions in the presence of a liquid-phase isomerization catalyst to produce an isomerized second side stream, and c) returning at least a portion of the isomerized second side stream to the second distillation column; and (X) optionally, conducting liquid-phase isomerization upon at least a portion of the second overhead stream external to the second distillation column under liquid-phase isomerization conditions in the presence of a liquid-phase isomerization catalyst to produce an isomerized second overhead stream; wherein liquid-phase isomerization is carried out in at least one of (II), (IV), (V), (VII), (IX), or (X), and the second overhead stream and/or the isomerized second overhead stream is rich in p-xylene. Liquid-phase isomerization at any of (II), (IV), (V), (VII), or (IX) may occur in the presence of C9+ aromatic hydrocarbons.

The feed mixture processed in accordance with the foregoing may comprise ethylbenzene in any amount, and at least a majority of the ethylbenzene may be obtained in the first overhead stream following distillation. Additional details regarding suitable feed mixtures and sources thereof is provided below.

Alternately, feed mixtures having ethylbenzene present below a specified amount, preferably feed mixture lean in ethylbenzene or lacking ethylbenzene, may be isomerized under liquid-phase isomerization conditions and further separated without first separating the feed mixture using a first distillation column. Such processes may comprise: (A) providing a feed mixture lean in ethylbenzene comprising one or more xylene isomers and C9+ aromatic hydrocarbons, o-xylene comprising at least a majority of the one or more xylene isomers; (B) optionally, conducting liquid-phase isomerization of the feed mixture under liquid-phase isomerization conditions in the presence of a liquid-phase isomerization catalyst to produce an isomerized feed mixture; (C) introducing the feed mixture or the isomerized feed mixture into a distillation column at a feed location thereon, the distillation column optionally containing a liquid-phase isomerization catalyst loaded therein; (D) optionally, contacting a portion of the feed mixture with the liquid-phase isomerization catalyst in the distillation column under liquid-phase isomerization conditions; (B) obtaining from the distillation column an overhead stream comprising at least o-xylene, a bottoms stream comprising C9+ aromatic hydrocarbons, and an optional side stream; (F) optionally, a) obtaining the side stream from the distillation column, b) conducting liquid-phase isomerization of the side stream external to the distillation column under liquid-phase isomerization conditions in the presence of a liquid-phase isomerization catalyst to produce an isomerized side stream, and c) returning at least a portion of the isomerized side stream to the distillation column; and (G) optionally, conducting liquid-phase isomerization upon at least a portion of the overhead stream external to the distillation column under liquid-phase isomerization conditions in the presence of a liquid-phase isomerization catalyst to produce an isomerized overhead stream; wherein liquid-phase isomerization is carried out in at least one of (B), (D), (F), or (G), and the overhead stream and/or the isomerized overhead stream is rich in p-xylene. Liquid-phase isomerization at any of (B), (D), or (F) may occur in the presence of C9+ aromatic hydrocarbons.

Suitable feed mixtures for use in the disclosure herein may include, but are not limited to, those obtained from a catalytic reforming process, a benzene or toluene alkylation process, a xylene isomerization process, a toluene disproportionation process, a transalkylation process, cracking (e.g., steam or catalytic cracking), a petroleum source, a bio-production source, or any combination thereof. The feed mixture may comprise ethylbenzene in an amount below a specified threshold amount or the feed mixture may be pre-processed/refined in a suitable manner to decrease the amount of ethylbenzene below a specified threshold amount needed to support a particular process configuration. In addition to one or more xylene isomers, the feed mixture may comprise ethylbenzene in an amount up to about 30 wt % or up to about 20 wt % of the total feed mixture. Preferably, the feed mixture may be produced or sourced with a low level of ethylbenzene to minimize the fraction of the feed mixture being processed by vapor-phase isomerization. For example, suitable feed mixtures may comprise ethylbenzene at about 2000 ppm or less, or about 1500 ppm or less, or about 1000 ppm or less based on total mass, or be further processed to afford an ethylbenzene concentration below these values. Particularly advantageous feed mixtures may be produced via toluene alkylation with methanol and/or dimethyl ether as an alkylation agent, which may afford p-xylene in considerably greater than equilibrium quantities relative to other xylene isomers, particularly o-xylene, as well as limit production of ethylbenzene (e.g., <2000 ppm by weight) and other problematic byproducts. Description of exemplary methylation catalysts, methylation agents, and methylation conditions for lower aromatic hydrocarbons may be found in, for example, U.S. Pat. Nos. 6,423,879; 6,504,072; 6,642,426; and 9,440,893, the relevant contents of which are incorporated herein by reference. In one example, a suitable feed mixture may comprise a raffinate stream rich in o-xylene obtained following separation of p-xylene produced in a toluene alkylation process with methanol and/or dimethyl ether.

Feed mixtures suitable for use in the disclosure herein may comprise one or more xylene isomers, optionally ethylbenzene, and C9+ aromatic hydrocarbons. The one or more xylene isomers may comprise an equilibrium or non-equilibrium distribution of o-xylene, m-xylene, and p-xylene. The feed mixture may be rich in any one of o-xylene, m-xylene, or p-xylene, relative to total xylene isomers, provided that sufficient o-xylene and C9+ aromatic hydrocarbons are present in the bottoms stream obtained from the first distillation column (or formable in the bottoms stream by liquid-phase isomerization) to facilitate separation in a second distillation column. In some embodiments, the feed mixture may comprise predominantly o-xylene, C9+ aromatic hydrocarbons, and optionally ethylbenzene.

In non-limiting examples, a total concentration of xylene isomers (including ethylbenzene) may range from c(xylenes)1 to c(xylenes)2 wt %, based on the total weight of the feed mixture, where c(xylenes)1 and c(xylenes)2 can be, independently, 30, 40, 50, 55, 60, 65, 70, 75, 80, 85, or 90, as long as c(xylenes)1<c(xylenes)2.

In non-limiting examples, a total concentration of p-xylene in the feed mixture may range from c(pX)1 to c(pX)2 wt %, based on the total weight of the feed mixture, where c(pX)1 and c(pX)2 can be, independently, 10, 20, 30, 40, 50, 55, 60, 65, 70, 75, 80, 85, or 90, as long as c(pX)1<c(pX)2.

In non-limiting examples, a total concentration of m-xylene in the feed mixture may range from c(mX)1 to c(mX)2 wt %, based on the total weight of the feed mixture, where c(mX)1 and c(mX)2 can be, independently, 10, 20, 30, 40, 50, 55, 60, 65, 70, 75, 80, 85, or 90, as long as c(mX)1<c(mX)2.

In non-limiting examples, a total concentration of o-xylene in the feed mixture may range from c(oX)1 to c(oX)2 wt %, based on the total weight of the feed mixture, where c(oX)1 and c(oX)2 can be, independently, 10, 20, 30, 40, 50, 55, 60, 65, 70, 75, 80, 85, or 90, as long as c(oX)1<c(oX)2.

In non-limiting examples, the feed mixture may comprise C9+ hydrocarbons, in total, in a range from c(C9+)1 to c(C9+)2 wt %, based on the total weight of the feed mixture, where c(C9+)1 and c(C9+)2 can be, independently, 0.01, 0.1, 1.0, 5.0, 10.0, 15.0, 20.0, 25.0, or 30.0 as long as c(C9+)1<c(C9+)2.

In non-limiting examples, the feed mixture may comprise ethylbenzene at a concentration ranging from c(EB)1 to c(EB)2 wt %, based on the total weight of the feed mixture, where c(EB)1 and c(EB)2 can be, independently, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, as long as c(EB)1<c(EB)2. Preferably, c(EB)2 is 20 wt % or below. More preferably, c(EB)2 is 10 wt % or below. More preferably, c(EB)2 is 5 wt % or below. Still more preferably, c(EB)2 is 2 wt % or below, or 1 wt % or below, more preferably about 2000 ppm or below, or about 1500 ppm or below, or about 1000 ppm or below. Optionally, a feed mixture having a higher ethylbenzene content may be further processed to achieve an ethylbenzene concentration in any of the foregoing ranges.

If the feed mixture is processed without being first separated in a first distillation column into an overhead stream and a bottoms stream, the feed mixture preferably has a low ethylbenzene concentration within the foregoing ranges. When the feed mixture is processed in this manner, the ethylbenzene content may be 2 wt % or below, or 1 wt % or below, more preferably about 2000 ppm or below, or about 1500 ppm or below, or about 1000 ppm or below.

The feed mixture may optionally comprise benzene and/or toluene. In non-limiting examples, the feed mixture may comprise benzene and toluene combined in a range from c(BT)1 to c(BT)2 wt %, based on the total weight of the feed mixture, where c(BT)1 and c(BT)2 can be, independently, 0.01, 0.1, 1.0, 2.0, 3.0, 5.0, 8.0, 10.0, 15.0, 20.0, 30.0, 40.0, or 50.0, as long as c(BT)1<c(BT)2. Preferably, c(BT)2 is 10.0 or less. More preferably, c(BT)2 is 5.0 or less. Still more preferably, c(BT)2 is 3.0 or less. In various embodiments, toluene may be the primary component between benzene and toluene, and in some embodiments, combined benzene and toluene may consist essentially of toluene. That is, in some embodiments, the feed mixture may be substantially free of benzene. In some embodiments, the feed mixture may be substantially free of toluene as well.

Accordingly, in some embodiments, the feed mixture may be rich in o-xylene and further contain C9+ aromatic hydrocarbons and optionally ethylbenzene. Optionally, such feed mixtures may further comprise m-xylene and/or p-xylene in individual amounts less than the amount of o-xylene or in a combined amount less than the amount of o-xylene. In other embodiments, suitable feed mixtures may be rich in p-xylene or m-xylene and further contain o-xylene, C9+ aromatic hydrocarbons, and optionally ethylbenzene. When the feed mixture is not initially separated in the first distillation column, ethylbenzene may still be present, albeit in a sufficiently low quantity to still facilitate p-xylene separation in a p-xylene recovery unit, as discussed above. Suitably low amounts of ethylbenzene are provided above.

Provided that liquid-phase isomerization has not been conducted within the first distillation column or upon a side stream removed from the first distillation column, the amount of p-xylene in the bottoms stream may range from c(pX)1 to c(pX)2 wt %, where c(pX)1 and c(pX)2 can be, independently, 0, 0.5, 1, 2, 3, 4, 5, 6, 7, 8, 9, or 10, as long as c(pX)1<c(pX)2. The total quantity of m-xylene in the bottoms stream obtained from the first distillation column may similarly range from c(mX)1 to c(mX)2 wt %, where c(mX)1 and c(mX)2 can be, independently, 0, 0.5, 1, 2, 3, 4, 5, 6, 7, 8, 9, or 10, as long as c(mX)1<c(mX)2. The total quantity of o-xylene in the bottoms stream may range from c(oX)1 to c(oX)2 wt %, where c(oX)1 and c(oX)2 can be, independently, 10, 15, 20, 25, 30, 35, 40, 45, 50, 55, 60, 65, or 70, as long as c(oX)1<c(oX)2.

In various embodiments of the processes disclosed herein, 80 wt % or greater, preferably 85 wt % or greater, more preferably 90 wt % or greater, more preferably 95 wt % or greater, more preferably 98 wt % or greater, more preferably 99 wt % or greater, or still more preferably approximately 100 wt % of the feed mixture may be in liquid-phase at the inlet of a liquid-phase isomerization unit in which the liquid-phase isomerization takes place. The feed mixture may have an inlet temperature in the range from T1 to T2° C., where T1 and T2 can be, independently, 200, 210, 220, 230, 240, 250, 255, 260, 265, 270, 275, 280, 285, 290, 295, or 300, as long as T1<T2. The relatively low inlet temperature of the feed mixture, in combination with other liquid-phase isomerization conditions described below may facilitate the liquid-phase isomerization of C8 aromatic hydrocarbons to form additional p-xylene.

Unless conducted in a distillation column, liquid-phase isomerization in the present disclosure may be conducted using a liquid-phase isomerization unit featuring a fixed bed reactor, a fluidized bed reactor, or a moving bed reactor. The feed provided to the liquid-phase isomerization conditions may be lean in p-xylene, in accordance with the disclosure above, and after liquid-phase isomerization, an equilibrium distribution of xylene isomers may be preferably obtained. If an equilibrium distribution of xylene isomers is not obtained, the liquid-phase isomerization conditions may be adjusted and/or liquid-phase isomerization may be repeated in a different location. The feed exposed to the liquid-phase isomerization conditions may flow upward, downward, or in a radial fashion within the liquid-phase isomerization unit. Alternately, liquid-phase isomerization may be conducted batchwise in a liquid-phase isomerization unit in some instances.

Suitable liquid-phase isomerization conditions may include a reaction gauge pressure in an isomerization unit ranging from p1 to p2 kPa, where p1 and p2 can be, independently, 1700, 1800, 1900, 2000, 2100, 2200, 2300, 2400, 2500, 2600, 2700, 2800, 2900, 3000, 3100, 3200, 3300, 3400, or 3500, as long as p1<p2. Preferably, p2 is 3000 kPa or lower. Preferably, p2 is 2500 kPa or lower. Higher reaction gauge pressures may promote dissolution of molecular hydrogen in the liquid-phase in the isomerization reaction, wherein the molecular hydrogen is provided as a co-feed in combination with the feed mixture to promote the liquid-phase isomerization reaction.

Suitable liquid-phase isomerization conditions may include a reaction temperature ranging from T1 to T2° C., where T1 and T2 can be, independently 200, 210, 220, 230, 240, 250, 255, 260, 265, 270, 275, 280, 285, 290, 295, or 300, as long as T1<T2. The relatively low reaction temperature during liquid-phase isomerization may improve energy efficiency by requiring less energy to heat the feed undergoing isomerization and by not requiring condensation of large quantities of a high-temperature vapor-phase following vapor-phase isomerization.

Suitable liquid-phase isomerization conditions may include a high WHSV ranging from w1 to w2 hour⁻¹, where w1 and w2 can be, 5.0, 5.5, 6.0, 6.5, 7.0, 7.5, 8.0, 8.5, 9.0, 9.5, 10, 11, 12, 12.5, 13, 14, 15, 16, 17, 17.5, 18, 19, or 20, as long as w1<w2. High WHSV values may be facilitated by co-feeding molecular hydrogen at a suitable rate.

Molecular hydrogen may be optionally provided as a co-feed to the liquid-phase isomerization conditions. In certain embodiments, the molecular hydrogen co-fed into an isomerization unit, or a portion thereof, can be introduced as a pressurized gas via an inlet upon the isomerization unit. Additionally or alternatively, the molecular hydrogen or a portion thereof can be fed into a feeding line, a vessel, or a storage tank associated with a feed provided to the liquid-phase isomerization conditions, which may promote admixture of the molecular hydrogen with the feed and deliver the molecular hydrogen to the liquid-phase isomerization conditions in combination with the feed. A majority (for example, ≥50%, ≥60%, ≥70%, ≥80%, ≥90%, ≥95%, ≥98%), more preferably substantially the entirety (≥99%), of the molecular hydrogen may be dissolved in the liquid-phase under the liquid-phase isomerization conditions. To achieve a higher concentration of dissolved molecular hydrogen in the liquid-phase, a suitably high pressure may be maintained in the isomerization unit.

In non-limiting examples, the molecular hydrogen can be fed into the isomerization unit at a feeding rate of r(H2)1 to r(H2)2 ppm by weight, based on the total weight of the feed, where r(H2)1 and r(H2)2 can be, independently, 100, 150, 200, 250, 300, 350, 400, 450, 500, 550, 600, 650, 700, 750, 800, 850, 950, 1000, 1500, 2000, 2500, 3000, 3500, 4000, 4500, or 5000, as long as r(H2)1<r(H2)2. Preferably, r(H2)2 is 3000 or less, 2000 or less, 1000 or less, 800 or less, 600 or less, or 500 or less.

Suitable liquid-phase isomerization catalysts may comprise a zeolite having an MEL framework structure (e.g., ZSM-11), an MFI framework structure (e.g., ZSM-5), or any combination thereof. Other suitable examples of zeolites that may be effective for conducting liquid-phase isomerization may include, for example, those having a MWW framework, a MOR framework, or the like. Examples may include: MWW-22, MWW-49, MWW-54, and combinations thereof.

In certain embodiments, the liquid-phase isomerization catalyst may comprise a first metal element selected from Fe, Co, Ni, Ru, Rh, Pd, Re, Os, Ir, Pt, and combinations thereof, and optionally a second metal selected from Sn, Zn, Ag, and combinations thereof. The first metal element may catalyze hydrogenation of olefins that may be produced under the liquid-phase isomerization, such as those produced by dealkylation of ethylbenzene. The second metal element may promote or enhance the catalytic effect of the first metal element. In other embodiments, the liquid-phase isomerization catalyst may be free of precious metal (i.e., Ru, Rh, Pd, Os, Ir, and Pt). In other embodiments, the liquid-phase isomerization catalyst may be free of any Group 7-10 metal. In still other embodiments, the liquid-phase isomerization catalyst may be free of any Group 7-15 metals except aluminum.

Zeolites having a MFI framework (e.g., ZSM-5) suitable for use in the present disclosure may have one or more of the following characteristics: presence in a hydrogen form (HZSM-5); a crystal size ≤0.1 micron; a mesoporous surface area (MSA) ≥45 m²/g; a total surface area to mesoporous surface area ratio ≤9; and a silica to alumina molar ratio in the range of 20 to 50.

Suitable zeolites having a MEL framework (e.g., ZSM-11) may comprise a plurality of primary crystallites, in which at least 75% (e.g., ≥80%, ≥85%, ≥90%, or even ≥95%) of the crystallites have crystallite size of less than or equal to 200 nanometer (e.g., ≤150, ≤100, ≤80, ≤50, ≤30 nanometers). Thus, at least 75% (e.g., ≥80%, ≥85%, ≥90%, or even ≥95%) of the crystallites may have a crystallite size in a range of es1 to cs2 nanometers (nm), where cs1 and cs2 can be, independently, 5, 10, 20, 30, 40, 50, 60, 70, 80, 90, 100, 120, 140, 150, 160, 180, or 200, as long as cs1<cs2. Preferably, cs1 is 10 or more and cs2 is 150 or less. More preferably, cs1 is 10 or more and cs2 is 50 or less. In this disclosure, crystallite size may be defined as the largest dimension of the crystallite observed under a transmission electron microscope (“TEM”). To determine crystallite size, a sample of the zeolite material is placed in a TEM, and an image of the sample is taken. The image is then analyzed to determine the crystallite size and distributions thereof. The small crystallite sizes of the MEL framework type zeolite material of this disclosure gives rise to surprisingly high catalytic activities and other advantages, in addition to the surprising tolerance toward limited reactivity of C9+ aromatic hydrocarbons under liquid-phase isomerization conditions.

Optionally, the primary crystallites of zeolites having a MEL framework may have an average primary crystallite size of less than 80 nm, preferably less than 70 nm, and in some cases less than 60 nm, in each of the a, b and c crystal vectors as measured by X-ray diffraction. The primary crystallites may optionally have an average primary crystallite size of greater than 20 nm, optionally greater than 30 nm, in each of the a, b and c crystal vectors, as measured by X-ray diffraction.

The primary crystallites may have a narrow particle size distribution such that at least 90% of the primary crystallites by number have a primary crystallite size in the range of from 10 to 80 nm, preferably in the range of from 20 to 50 nm, as determined by analysis of images of the primary crystallites taken by TEM.

Crystallites of zeolites having a MEL framework may assume various shapes such as substantially spherical, rod-like, or the like. Alternately or in addition, the crystallites can have irregular shapes in TEM images. Thus, a crystallite may exhibit a longest dimension in a first direction (“primary dimension”), and a width in another direction perpendicular to the first direction (“secondary dimension”), where the width is defined as the dimension in the middle of the primary dimension, as determined by TEM image analysis. The ratio of the primary dimension to the width is called the aspect ratio of the crystallite. In certain embodiments, the crystallites can have an average aspect ratio determined by TEM image analysis in a range from ar1 to ar2, where ar1 and ar2 can be, independently, 1, 1.2, 1.4, 1.5, 1.6, 1.8, 2.0, 2.2, 2.4, 2.5, 2.6, 2.8, 3.0, 3.2, 3.4, 3.5, 3.6, 3.8, 4.0, 4.2, 4.4, 4.5, 4.6, 4.7, 4.8, or 5.0, as long as ar1<ar2. Preferably, ar1 is 1 or greater and ar2 is 3 or less, or ar1 is 1 or greater and ar2 is 2 or less.

The small crystallites of zeolites having a MEL framework may aggregate to form agglomerates. The agglomerates are polycrystalline materials having void space at the boundary of the crystallites. The agglomerates may be formed from primary crystallites having an average primary crystallite size as determined by TEM image analysis of less than 80 nm, preferably less than 70 nm and more preferably less than 60 nm, or even less than 50 nm.

Suitable zeolites having a MEL framework may comprise a mixture of agglomerates of the primary crystallites together with some unagglomerated primary crystallites. The majority of the zeolites having a MEL framework may comprise, for example, greater than 50 wt % or greater than 80 wt % may comprise agglomerates of primary crystallites. The agglomerates can be regular or irregular form. For more information on agglomerates please see Walter, D. (2013) Primary Particles—Agglomerates—Aggregates, in Nanomaterials (ed Deutsche Forschungsgemeinschaft (DFG)), Wiley-VCH Verlag GmbH & Co. KGaA, Weinheim, Germany. doi: 10.1002/9783527673919, pages 1-24.

Preferably, zeolites having a MEL framework may comprise less than 10% by weight of primary crystallites having a size of >200 nm as determined by TEM image analysis, or less than 10% by weight of primary crystallites having a size of >150 nm as determined by TEM image analysis, or less than 10% by weight of primary crystallites having a size of >100 nm as determined by TEM image analysis, or less than 10% by weight of primary crystallites having a size of >80 nm as determined by TEM image analysis.

Suitable zeolites having a MEL framework may have a silica to alumina ratio of R(s/a) that can vary from r1 to r2, where r1 and r2 can be, independently, 10, 12, 14, 15, 16, 18, 20, 22, 24, 25, 26, 28, 30, 32, 34, 35, 36, 38, 40, 42, 44, 45, 46, 48, 50, 52, 54, 55, 56, 58, or 60, as long as r1<r2. Preferably, r1 is 20 or greater and r2 is 50 or less. Preferably, r1 is 20 or greater and r2 is 40 or less. Preferably, r1 is 20 or greater and r2 is 30 or less. Ratio R(s/a) can be determined by ICP-MS (inductively coupled plasma mass spectrometry) or XRF (X-ray fluorescence).

Suitable zeolites having a MEL framework may have a BET total specific surface area of A(st) that can vary from a1 to a2 m²/g, where a1 and a2 can be, independently, 300, 320, 340, 350, 360, 380, 400, 420, 440, 450, 460, 480, 500, 520, 540, 550, 560, 580, or 600, as long as a1<a2. Preferably, a1 is 400 or greater and a2 is 500 or less. Preferably, al is 400 or greater and a2 is 475 or less. A(st) can be determined by the BET method (Brunauer-Emmet-Teller method, a nitrogen adsorption method). The high total surface area A(st) of the zeolite material of this disclosure is another reason why it exhibits high catalytic activity for converting C8 aromatic hydrocarbons. The BET method can yield a total specific area of a measured material, including a microporous specific area component and a mesopore specific area component. The mesopore specific area may be called mesopore area, mesoporous area, or external area in this disclosure. The total specific area may be called total surface area or total area in this disclosure.

Suitable zeolites having a MEL framework may have a mesopore area of A(mp) that is ≥15% (e.g., ≥16%, ≥18%, ≥20%, ≥22%, ≥24%, ≥25%) of the total surface area A(st) discussed above. In certain embodiments it is preferred that A(mp)≥20%*A(st). In certain embodiments, it is preferred that A(mp)≤40%*A(st). In certain embodiments, it is preferred that A(mp)≤30%*A(st). The high mesopore area A(mp) of the zeolite material of this disclosure is another reason why it exhibits a high catalytic activity for converting aromatic hydrocarbons. Without intending to be bound by a particular theory, it is believed that the catalystic sites present on the mesopore area of the zeolite material of this disclosure are more numerous due to the high mesopore area, which tend to contribute more to the catalytic activity than are catalytic sites located in deep channels inside the zeolite material. The time required for reactant molecules to reach the catalytic sites on the mesopore surfaces and the product molecules to exit them is relatively short. Conversely, it would take significantly longer time for reactant molecules to diffuse into deep channels and for the product molecules to diffuse out of them.

Suitable zeolites having a MEL framework may have a hexane sorption value of v(hs) that can vary from v1 to v2 mg/g, where v1 and v2 can be, independently, 90, 92, 94, 95, 96, 98, 100, 102, 104, 105, 106, 108, or 110, as long as v1<v2. Hexane sorption value can be determined by TGA (thermogravimetric analysis) as is typical in the industry.

Suitable zeolites having a MEL framework may have an alpha value that can vary from a1 to a2, where a1 and a2 can be, independently, 500, 600, 700, 800, 900, 1000, 1200, 1400, 1500, 1600, 1800, 2000, 2200, 2400, 2500, 2600, 2800, or 3000, as long as a1<a2 Alpha value can be determined by the method described in U.S. Pat. No. 3,354,078 and Journal of Catalysis, Vol. 4, p. 527 (1965); vol. 6, p. 278 (1966) and Vol. 61, p. 395 (1980).

Optionally, suitable zeolites having a MEL framework may be calcined and subjected to post-treatments such as steaming and/or acid washing. Steaming may be conducted at a temperature of at least 200° C., preferably at least 350° C., more preferably at least 400° C., in some cases at least 500° C., for a period of from 1 to 20 hours, preferably from 2 to 10 hours. Acid washing may be conducted with an aqueous solution of an acid, preferably an organic acid, such as a carboxylic acid, preferably oxalic acid. Optionally, a steamed zeolite may be treated with an aqueous solution of an acid at a temperature of at least 50° C., preferably at least 60° C., for a period of at least 1 hour, preferably at least 4 hours, for example, in the range of from 5 to 20 hours. Preferably, a treated zeolite having a MEL framework may have a chemical composition with a molar ratio of nSiO₂:Al₂O₃, wherein n is at least 20, more preferably at least 50, and in some cases at least 100.

The liquid-phase isomerization catalysts suitable for use in the disclosure herein may be formulated with a binder or present as an unbound free powder. The binder may comprise a binder material resistant to the temperature and other liquid-phase isomerization conditions. Examples of suitable binder materials include clays, alumina, silica, silica-alumina, silica-magnesia, silica-zirconia, silica-thoria, silica-beryllia, and silica-titania, as well as ternary compositions, such as silica-alumina-thoria, silica-alumina-zirconia, silica-alumina-magnesia and silica-magnesia-zirconia. In non-limiting examples, a binder material may be included with the liquid-phase isomerization catalyst at a concentration from c1 to c2 wt %, based on the total weight of the catalyst, where c1 and c2 can be, independently, 1, 5, 10, 15, 20, 25, 30, 35, 40, 45, 50, 55, 60, 65, 70, 75, 80, 85, 90, 95, 98, 99, as long as c1<c2. The inclusion of a binder in the isomerization catalyst can enhance its mechanical strength, among other factors. A zeolite capable of promoting liquid-phase isomerization may also be blended with a second zeolite as a binder material, thereby forming a zeolite-bound zeolite, as described in U.S. Pat. Nos. 5,993,642 and 5,994,603 and each incorporated herein by reference. The relative proportions of zeolite and binder material may range from about 1:99 to about 99:1 on a mass basis. In illustrative examples, the zeolite capable of promoting liquid-phase isomerization may be present in an amount of 10% to about 70% by mass of the zeolite-bound zeolite, or about 20% to about 50% by mass of the zeolite-bound zeolite.

The liquid-phase isomerization catalyst may be a freshly made catalyst, a regenerated catalyst, or a mixture thereof. Regeneration of the catalyst may be conducted in the isomerization unit after the catalyst activity has decreased to a threshold level at the end of catalyst cycle, such as by exposing the catalyst to a stream of gas comprising molecular hydrogen. Alternatively, ex situ regeneration of the catalyst may be implemented, where the spent catalyst is taken out of the isomerization unit, heated in an oxygen-rich environment and/or exposed to a gas stream comprising molecular hydrogen to abate coke on its surface.

After conducting liquid-phase isomerization in accordance with the disclosure above, processes of the present disclosure may further comprise separating at least a portion of the p-xylene using a p-xylene recovery unit. More specifically, when two distillation columns are used to process a feed mixture, processes of the present disclosure may further comprise: (XI) feeding at least a portion of the second overhead stream and/or the isomerized second overhead stream into a p-xylene recovery unit; and (XII) obtaining a first stream rich in p-xylene and a second stream lean in p-xylene from the p-xylene recovery unit. Similarly, when one distillation column is used to process a feed mixture lean in ethylbenzene, the process may further comprise: (H) feeding at least a portion of the overhead stream and/or the isomerized overhead stream into a p-xylene recovery unit; and (I) obtaining a first stream rich in p-xylene and a second stream lean in p-xylene from the p-xylene recovery unit. The p-xylene recovery unit may utilize any technique suitable for separating a first stream sufficiently rich in p-xylene. Suitable separation techniques may include adsorption chromatography (e.g., simulated moving bed chromatography), crystallization (e.g., fractional crystallization), or any combination thereof.

### Vapor-Phase Isomerization

In any of the foregoing embodiments, the second stream lean in p-xylene that is obtained from the p-xylene recovery unit may be further processed by vapor-phase isomerization to produce additional p-xylene. Optionally, the first overhead stream (where obtained) may be combined with the second stream lean in p-xylene and subjected to vapor-phase isomerization as well, which may isomerize ethylbenzene or other byproducts present therein and also form additional p-xylene. Such vapor-phase isomerization may be conducted under vapor-phase isomerization conditions in the presence of a suitable vapor-phase isomerization catalyst, as described in further detail hereinafter.

Suitable vapor-phase isomerization conditions may include a temperature and a pressure such that a majority of the xylenes are in a vapor-phase. Description of exemplary vapor-phase isomerization processes, conditions, and catalysts can be found in, for example, U.S. Patent Application Publications 2011/03196881; 2012/0108867; 2012/0108868; 2014/0023563; 2015/0051430; and 2017/0081259, the relevant contents of which are incorporated herein by reference.

In one example, suitable vapor-phase isomerization catalyst may include zeolites having a MWW framework. Such zeolites may have a Constraint Index ≤5 and include molecular sieves having one or more of the following properties:
a) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, incorporated herein by reference);
b) molecular sieves made from a second degree building block, being a 2-dimensional tiling of such MWW framework topology unit cells, forming a monolayer of one unit cell thickness, in an embodiment, one c-unit cell thickness;
c) molecular sieves made from common second degree building blocks, being layers of one or more than one unit cell thickness, where the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
d) molecular sieves made by any regular or random 2-dimensional or 3-dimensional combination of unit cells having the MWW framework topology.

Example zeolites having a MWW framework include MCM-22 (U.S. Pat. No. 4,954,325), PSH-3 (U.S. Pat. No. 4,439,409), SSZ-25 (U.S. Pat. No. 4,826,667), ERB-1 (European Patent No. 0293032), ITQ-1 (U.S. Pat. No. 6,077,498), ITQ-2 (International Publication No. WO97/17290), MCM-36 (U.S. Pat. No. 5,250,277), MCM-49 (U.S. Pat. No. 5,236,575), MCM-56 (U.S. Pat. No. 5,362,697), UZM-8 (U.S. Pat. No. 6,756,030), UZM-8HS (U.S. Pat. No. 7,713,513), UZM-37 (U.S. Pat. No. 7,982,084), EMM-10 (U.S. Pat. No. 7,842,277), EMM-12 (U.S. Pat. No. 8,704,025), EMM-13 (U.S. Pat. No. 8,704,023), UCB-3 (U.S. Pat. No. 9,790,143B2) and mixtures thereof.

In some embodiments, the zeolites having a MWW framework may be contaminated with other crystalline materials, such as ferrierite or quartz, which may be present in quantities of ≤10 wt % or ≤5 wt %.

The present disclosure further relates to the following non-limiting aspects and/or embodiments:

A1. A process comprising:
(I) introducing a feed mixture comprising one or more xylene isomers and C9+ aromatic hydrocarbons into a first distillation column at a feed location thereon, the first distillation column optionally containing a liquid-phase isomerization catalyst loaded therein;
(II) optionally, contacting a portion of the feed mixture with the liquid-phase isomerization catalyst in the first distillation column under liquid-phase isomerization conditions;
(III) obtaining from the first distillation column a first overhead stream comprising m-xylene, p-xylene, or any combination thereof, a first bottoms stream comprising o-xylene and C9+ aromatic hydrocarbons, and an optional first side stream;
(IV) optionally, a) obtaining the first side stream from the first distillation column, b) conducting liquid-phase isomerization of the first side stream external to the first distillation column under liquid-phase isomerization conditions in the presence of a liquid-phase isomerization catalyst to produce an isomerized first side stream, and c) returning at least a portion of the isomerized first side stream to the first distillation column;
(V) optionally, conducting liquid-phase isomerization upon at least a portion of the first bottoms stream external to the first distillation column under liquid-phase isomerization conditions in the presence of a liquid-phase isomerization catalyst to produce an isomerized first bottoms stream;
(VI) feeding at least a portion of the first bottoms stream and/or at least a portion of the isomerized first bottoms stream to a second distillation column optionally containing a liquid-phase isomerization catalyst loaded therein,
(VII) optionally, contacting a portion of the first bottoms stream with the liquid-phase isomerization catalyst in the second distillation column under liquid-phase isomerization conditions;
(VIII) obtaining from the second distillation column a second overhead stream comprising at least o-xylene, a second bottoms stream comprising C9+ aromatic hydrocarbons, and an optional second side stream;
(IX) optionally, a) obtaining the second side stream from the second distillation column, b) conducting liquid-phase isomerization of the second side stream external to the second distillation column under liquid-phase isomerization conditions in the presence of a liquid-phase isomerization catalyst to produce an isomerized second side stream, and c) returning at least a portion of the isomerized second side stream to the second distillation column; and
(X) optionally, conducting liquid-phase isomerization upon at least a portion of the second overhead stream external to the second distillation column under liquid-phase isomerization conditions in the presence of a liquid-phase isomerization catalyst to produce an isomerized second overhead stream;
   wherein liquid-phase isomerization is carried out in at least one of (II), (IV), (V), (VII), (IX), or (X), and the second overhead stream and/or the isomerized second overhead stream is rich in p-xylene.

A2. The process of A1, further comprising:
(XI) feeding at least a portion of the second overhead stream and/or the isomerized second overhead stream to a p-xylene recovery unit; and
(XII) obtaining a first stream rich in p-xylene and a second stream lean in p-xylene from the p-xylene recovery unit.

A3. The process of A2, wherein the p-xylene recovery unit separates the first stream from the second stream by adsorption chromatography, crystallization, or any combination thereof.

A4. The process of any one of A1-A3, wherein the liquid-phase isomerization catalyst comprises a zeolite having a MEL framework structure, a zeolite having a MFI framework structure, or any combination thereof.

A5. The process of any one of A1-A4, wherein liquid-phase isomerization is carried out in (II).

A6. The process of any one of A1-A4, wherein liquid-phase isomerization is carried out in (IV).

A7. The process of any one of A1-A4, wherein liquid-phase isomerization is carried out in (V).

A8. The process of any one of A1-A4, wherein liquid-phase isomerization is carried out in (VII).

A9. The process of any one of A1-A4, wherein liquid-phase isomerization is carried out in (IX).

A10. The process of any one of A1-A4, wherein liquid-phase isomerization is carried out in (X).

A11. The process of any one of A1-A10, wherein liquid-phase isomerization is carried out in one of (II), (IV), (V), (VII), (IX), or (X).

A12. The process of any one of A1-A4, wherein liquid-phase isomerization is carried out in at least one of (IV), (V), (IX), or (X) in at least one liquid-phase isomerization unit.

A13. The process of A12, wherein liquid-phase isomerization is carried out in one of (IV), (V), (IX), or (X).

A14. The process of A12 or A13, further comprising:

at least temporarily bypassing the at least one liquid-phase isomerization unit.

A15. The process of any one of A1-A4, wherein liquid-phase isomerization is carried out in at least one of (II), (IV), (V), (VII), or (IX) in the presence of C9+ aromatic hydrocarbons.

A16. The process of A15, wherein liquid-phase isomerization is carried out in one of (II), (IV), (V), (VII), or (IX) in the presence of C9+ aromatic hydrocarbons.

A17. The process of any one of A2-A16, further comprising:

conducting vapor-phase isomerization under vapor-phase isomerization conditions in the presence of a vapor-phase isomerization catalyst upon at least a portion of the second stream, optionally wherein the first overhead stream and the second stream are combined with one another before undergoing vapor-phase isomerization.

A18. The process of any one of A1-A17, wherein the feed mixture comprises ethylbenzene and at least a majority of the ethylbenzene is obtained in the first overhead stream.

A19. The process of any one of A1-A18, wherein o-xylene comprises at least a majority of the one or more xylene isomers in the feed mixture.

B1. A process comprising:
(A) providing a feed mixture comprising one or more xylene isomers and C9+ aromatic hydrocarbons, o-xylene comprising at least a majority of the one or more xylene isomers, and the feed mixture being lean in ethylbenzene;
(B) optionally, conducting liquid-phase isomerization upon at least a portion of the feed mixture under liquid-phase isomerization conditions in the presence of a liquid-phase isomerization catalyst to produce an isomerized feed mixture;
(C) introducing the feed mixture and/or at least a portion of the isomerized feed mixture into a distillation column at a feed location thereon, the distillation column optionally containing a liquid-phase isomerization catalyst loaded therein;
(D) optionally, contacting a portion of the feed mixture with the liquid-phase isomerization catalyst in the distillation column under liquid-phase isomerization conditions;
(E) obtaining from the distillation column an overhead stream comprising at least o-xylene, a bottoms stream comprising C9+ aromatic hydrocarbons, and an optional side stream;
(F) optionally, a) obtaining the side stream from the distillation column, b) conducting liquid-phase isomerization of the side stream external to the distillation column under liquid-phase isomerization conditions in the presence of a liquid-phase isomerization catalyst to produce an isomerized side stream, and c) returning at least a portion of the isomerized side stream to the distillation column;
(G) optionally, conducting liquid-phase isomerization upon at least a portion of the overhead stream external to the distillation column under liquid-phase isomerization conditions in the presence of a liquid-phase isomerization catalyst to produce an isomerized overhead stream;
   wherein liquid-phase isomerization is carried out in at least one of (B), (D), (F), or (G), and the overhead stream and/or the isomerized overhead stream is rich in p-xylene.

B2. The process of BI, further comprising:
(H) feeding at least a portion of the overhead stream and/or the isomerized overhead stream into a p-xylene recovery unit; and
(I) obtaining a first stream rich in p-xylene and a second stream lean in p-xylene from the p-xylene recovery unit.

B3. The process of B2, wherein the p-xylene recovery unit separates the first stream from the second stream by adsorption chromatography, crystallization, or any combination thereof.

B4. The process of any one of B1-B3, wherein the liquid-phase isomerization catalyst comprises a zeolite having a MEL framework structure, a zeolite having a MFI framework structure, or any combination thereof.

B5. The process of any one of B1-B4, wherein liquid-phase isomerization is carried out in one of (B), (D), (F), or (G).

B6. The process of any one of B1-B4, wherein liquid-phase isomerization is carried out in one of (B), (D), or (F) in the presence of C9+ aromatic hydrocarbons.

B7. The process of any one of B1-B6, further comprising:

conducting vapor-phase isomerization under vapor-phase isomerization conditions in the presence of a vapor-phase isomerization catalyst upon at least a portion of the second stream and/or feeding at least a portion of the second stream to the distillation column.

To facilitate a better understanding of the embodiments of the present disclosure, the following examples of preferred or representative embodiments are given. In no way should the following examples be read to limit, or to define, the scope of the invention.

### EXAMPLES

A catalyst substantially inert to toluene conversion under liquid-phase isomerization conditions was prepared as described in U.S. Patent Application Publication 2022/0134318. The catalyst had a ZSM-11 zeolite framework with a Si:Al2 ratio of 25:1.

A commercial C9+ heavy aromatics stream was mixed with o-xylene to afford a 1:1 wt/wt mixture. The mixture was reacted under various liquid-phase isomerization conditions specified in Table 1 below using the above catalyst.

TABLE 1

| | Run 1 | Run 2 | Run 3 | Run 4 |
|---|---|---|---|---|
| Pressure (psig) | 264 | 264 | 264 | 264 |
| H2 (ppm) | 0 | 0 | 0 | 0 |
| WHSV (hour⁻¹) | 5.6 | 5.6 | 5.6 | 11 |
| Temperature (° C.) | 250 | 260 | 280 | 280 |
| p-Xylene Selectivity | 10.6 | 15.9 | 22.8 | 20.7 |
| in Product (wt %) | | | | |
| Benzene Yield (wt %) | 0.29 | 0.34 | 0.5 | 0.44 |
| Toluene Yield (wt %) | 0.41 | 0.62 | 1.4 | 1.1 |
| Ethylbenzene Yield (wt %) | 0.04 | 0.06 | 0.14 | 0.11 |
| C9+ Aromatic | ~1 | ~1 | ~1 | ~1 |
| Hydrocarbon Conversion (%) | | | | |
| Xylene Loss (wt %) | ~0.5 | ~1 | ~2.5 | ~2 |

Under all the conditions shown above, the reaction remained substantially in a liquid phase, since vaporization begins at approximately 300° C. As shown, there was minimal conversion of C9+ aromatic hydrocarbons or xylene loss under the liquid-phase isomerization reaction conditions, while the conversion of o-xylene still remained high.

Many alterations, modifications, and variations will be apparent to one having ordinary skill in the art in light of the foregoing description without departing from the spirit or scope of the present disclosure and that when numerical limits and numerical upper limits are listed herein, ranges from any lower limit to any upper limit are contemplated.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed, including the lower limit and upper limit. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

1. A process comprising:
(I) introducing a feed mixture comprising one or more xylene isomers and C9+ aromatic hydrocarbons into a first distillation column at a feed location thereon, the first distillation column optionally containing a liquid-phase isomerization catalyst loaded therein;
(II) optionally, contacting a portion of the feed mixture with the liquid-phase isomerization catalyst in the first distillation column under liquid-phase isomerization conditions;
(III) obtaining from the first distillation column a first overhead stream comprising m-xylene, p-xylene, or any combination thereof, a first bottoms stream comprising o-xylene and C9+ aromatic hydrocarbons, and an optional first side stream;
(IV) optionally, a) obtaining the first side stream from the first distillation column, b) conducting liquid-phase isomerization of the first side stream external to the first distillation column under liquid-phase isomerization conditions in the presence of a liquid-phase isomerization catalyst to produce an isomerized first side stream, and c) returning at least a portion of the isomerized first side stream to the first distillation column;
(V) optionally, conducting liquid-phase isomerization upon at least a portion of the first bottoms stream external to the first distillation column under liquid-phase isomerization conditions in the presence of a liquid-phase isomerization catalyst to produce an isomerized first bottoms stream;
(VI) feeding at least a portion of the first bottoms stream and/or at least a portion of the isomerized first bottoms stream to a second distillation column optionally containing a liquid-phase isomerization catalyst loaded therein;
(VII) optionally, contacting a portion of the first bottoms stream with the liquid-phase isomerization catalyst in the second distillation column under liquid-phase isomerization conditions;
(VIII) obtaining from the second distillation column a second overhead stream comprising at least o-xylene, a second bottoms stream comprising C9+ aromatic hydrocarbons, and an optional second side stream;
(IX) optionally, a) obtaining the second side stream from the second distillation column, b) conducting liquid-phase isomerization of the second side stream external to the second distillation column under liquid-phase isomerization conditions in the presence of a liquid-phase isomerization catalyst to produce an isomerized second side stream, and c) returning at least a portion of the isomerized second side stream to the second distillation column; and
(X) optionally, conducting liquid-phase isomerization upon at least a portion of the second overhead stream external to the second distillation column under liquid-phase isomerization conditions in the presence of a liquid-phase isomerization catalyst to produce an isomerized second overhead stream;
wherein liquid-phase isomerization is carried out in at least one of (II), (IV), (V), (VII), (IX), and (X), and the second overhead stream and/or the isomerized second overhead stream is rich in p-xylene.

2. The process of claim 1, further comprising:
(XI) feeding at least a portion of the second overhead stream and/or the isomerized second overhead stream to a p-xylene recovery unit; and
(XII) obtaining a first stream rich in p-xylene and a second stream lean in p-xylene from the p-xylene recovery unit.

3. The process of claim 2, wherein the p-xylene recovery unit separates the first stream from the second stream by adsorption chromatography, crystallization, or any combination thereof.

4. The process of claim 2, further comprising:
conducting vapor-phase isomerization under vapor-phase isomerization conditions in the presence of a vapor-phase isomerization catalyst upon at least a portion of the second stream, optionally wherein the first overhead stream and the second stream are combined with one another before undergoing vapor-phase isomerization.

5. The process of claim 1, wherein the liquid-phase isomerization catalyst comprises a zeolite having a MEL framework structure, a zeolite having a MFI framework structure, or any combination thereof.

6. The process of claim 1, wherein liquid-phase isomerization is carried out in (II).

7. The process of claim 1, wherein liquid-phase isomerization is carried out in (IV).

8. The process of claim 1, wherein liquid-phase isomerization is carried out in (V).

9. The process of claim 1, wherein liquid-phase isomerization is carried out in (VII).

10. The process of claim 1, wherein liquid-phase isomerization is carried out in (IX).

11. The process of claim 1, wherein liquid-phase isomerization is carried out in (X).

12. The process of claim 1, wherein liquid-phase isomerization is carried out in one of (II), (IV), (V), (VII), (IX), or (X).

13. The process of claim 1, wherein liquid-phase isomerization is carried out in at least one of (IV), (V), (IX), or (X) in at least one liquid-phase isomerization unit.

14. The process of claim 13, wherein liquid-phase isomerization is carried out in one of (IV), (V), (IX), or (X).

15. The process of claim 14, further comprising:
at least temporarily bypassing the at least one liquid-phase isomerization unit.

16. The process of claim 1, wherein liquid-phase isomerization is carried out in at least one of (II), (IV), (V), (VII), or (IX) in the presence of C9+ aromatic hydrocarbons.

17. The process of claim 16, wherein liquid-phase isomerization is carried out in one of (II), (IV), (V), (VII), or (IX) in the presence of C9+ aromatic hydrocarbons.

18. The process of claim 1, wherein the feed mixture comprises ethylbenzene and at least a majority of the ethylbenzene is obtained in the first overhead stream.

19. The process of claim 1, wherein o-xylene comprises at least a majority of the one or more xylene isomers in the feed mixture.

20. A process comprising:
(A) providing a feed mixture comprising one or more xylene isomers and C9+ aromatic hydrocarbons, o-xylene comprising at least a majority of the one or more xylene isomers, and the feed mixture being lean in ethylbenzene;
(B) optionally, conducting liquid-phase isomerization upon at least a portion of the feed mixture under liquid-phase isomerization conditions in the presence of a liquid-phase isomerization catalyst to produce an isomerized feed mixture;
(C) introducing the feed mixture and/or at least a portion of the isomerized feed mixture into a distillation column at a feed location thereon, the distillation column optionally containing a liquid-phase isomerization catalyst loaded therein;
(D) optionally, contacting a portion of the feed mixture with the liquid-phase isomerization catalyst in the distillation column under liquid-phase isomerization conditions;
(E) obtaining from the distillation column an overhead stream comprising at least o-xylene, a bottoms stream comprising C9+ aromatic hydrocarbons, and an optional side stream;
(F) optionally, a) obtaining the side stream from the distillation column, b) conducting liquid-phase isomerization of the side stream external to the distillation column under liquid-phase isomerization conditions in the presence of a liquid-phase isomerization catalyst to produce an isomerized side stream, and c) returning at least a portion of the isomerized side stream to the distillation column;
(G) optionally, conducting liquid-phase isomerization upon at least a portion of the overhead stream external to the distillation column under liquid-phase isomerization conditions in the presence of a liquid-phase isomerization catalyst to produce an isomerized overhead stream;
wherein liquid-phase isomerization is carried out in at least one of (B), (D), (F), or (G), and the overhead stream and/or the isomerized overhead stream is rich in p-xylene.

21. The process of claim 20, further comprising:
(H) feeding at least a portion of the overhead stream and/or the isomerized overhead stream into a p-xylene recovery unit; and
(I) obtaining a first stream rich in p-xylene and a second stream lean in p-xylene from the p-xylene recovery unit.

22. The process of claim 21, wherein the p-xylene recovery unit separates the first stream from the second stream by adsorption chromatography, crystallization, or any combination thereof.

23. The process of claim 22, wherein the liquid-phase isomerization catalyst comprises a zeolite having a MEL framework structure, a zeolite having a MFI framework structure, or any combination thereof.

24. The process of claim 20, wherein liquid-phase isomerization is carried out in one of (B), (D), (F), or (G).

25. The process of claim 20, wherein liquid-phase isomerization is carried out in one of (B), (D), or (F) in the presence of C9+ aromatic hydrocarbons.

26. The process of claim 20, further comprising:
conducting vapor-phase isomerization under vapor-phase isomerization conditions in the presence of a vapor-phase isomerization catalyst upon at least a portion of the second stream and/or feeding at least a portion of the second stream to the distillation column.
